SEQUENCE LISTING (55 sequences)

SEQ ID NO: 1 - protein, 200 aa, Artificial Sequence
  Synthetic
MASLSAVVVAPSVSSSAAVPPAPPLPGDSGTVIPPPPPPPPLPGGVVPPSPPLPPGTCIPPPPPLPGGACIPPPPQLPGSAAIPPPPPLPGVASIPPPPPLPGATAIPPPPPLPGATAIPPPPPLPGGTGIPPPPPPLPGSVGVPPPPPLPGGPGLPPPPPPFPGAPGIPPPPPGMGVPPPPPFGFGVPAAPVLPFGLTP

SEQ ID NO: 2 - protein, 431 aa, Artificial Sequence
  Synthetic
KKVYKPEVQLRRPNWSKFVAEDLSQDCFWTKVKEDRFENNELFAKLTLAFSAQTKTSKAKKDQEGGEEKKSVQKKKVKELKVLDSKTAQNLSIFLGSFRMPYQEIKNVILEVNEAVLTESMIQNLIKQMPEPEQLKMLSELKEEYDDLAESEQFGVVMGTVPRLRPRLNAILFKLQFSEQVENIKPEIVSVTAACEELRKSENFSSLLELTLLVGNYMNAGSRNAGAFGFNISFLCKLRDTKSADQKMTLLHFLAELCENDHPEVLKFPDELAHVEKASRVSAENLQKSLDQMKKQIADVERDVQNFPAATDEKDKFVEKMTSFVKDAQEQYNKLRMMHSNMETLYKELGDYFVFDPKKLSVEEFFMDLHNFRNMFLQAVKENQKRRETEEKMRRAKLAKEKAEKERLEKQQKREQLIDMNAEGDETGVMD

SEQ ID NO: 3 - protein, 631 aa, Artificial Sequence
  Synthetic
MASLSAVVVAPSVSSSAAVPPAPPLPGDSGTVIPPPPPPPPLPGGVVPPSPPLPPGTCIPPPPPLPGGACIPPPPQLPGSAAIPPPPPLPGVASIPPPPPLPGATAIPPPPPLPGATAIPPPPPLPGGTGIPPPPPPLPGSVGVPPPPPLPGGPGLPPPPPPFPGAPGIPPPPPGMGVPPPPPFGFGVPAAPVLPFGLTPKKVYKPEVQLRRPNWSKFVAEDLSQDCFWTKVKEDRFENNELFAKLTLAFSAQTKTSKAKKDQEGGEEKKSVQKKKVKELKVLDSKTAQNLSIFLGSFRMPYQEIKNVILEVNEAVLTESMIQNLIKQMPEPEQLKMLSELKEEYDDLAESEQFGVVMGTVPRLRPRLNAILFKLQFSEQVENIKPEIVSVTAACEELRKSENFSSLLELTLLVGNYMNAGSRNAGAFGFNISFLCKLRDTKSADQKMTLLHFLAELCENDHPEVLKFPDELAHVEKASRVSAENLQKSLDQMKKQIADVERDVQNFPAATDEKDKFVEKMTSFVKDAQEQYNKLRMMHSNMETLYKELGDYFVFDPKKLSVEEFFMDLHNFRNMFLQAVKENQKRRETEEKMRRAKLAKEKAEKERLEKQQKREQLIDMNAEGDETGVMD

SEQ ID NO: 4 - protein, 1419 aa, Artificial Sequence
  Synthetic
MEGTHCTLQLHKPITELCYISFCLPKGEVRGFSYKGTVTLDRSNKGFHNCYQVREESDIISLSQEPDEHPGDIFFKQTPTKDILTELYKLTTERERLLTNLLSSDHILGITMGNQEGKLQELSVSLAPEDDCFQSAGDWQGELPVGPLNKRSTHGNKKPRRSSGRRESFGALPQKRTKRKGRGGRESAPLMGKDKICSSHSLPLSRTRPNLWVLEEKGNLLPNGALACSLQRRESCPPDIPKTPDTDLGFGSFETAFKDTGLGREVLPPDCSSTEAGGDGIRRPPSGLEHQQTGLSESHQDPEKHPEAEKDEMEKPAKRTCKQKPVSKVVAKVQDLSSQVQRVVKTHSKGKETIAIRPAAHAEFVPKADLLTLPGAEAGAHGSRRQGKERQGDRSSQSPAGETASISSVSASAEGAVNKVPLKVIESEKLDEAPEGKRLGFPVHTSVPHTRPETRNKRRAGLPLGGHKSLFLDLPHKVGPDSSQPRGDKKKPSPPAPAALGKVFNNSASQSSTHKQTSPVPSPLSPRLPSPQQHHRILRLPALPGEREAALNDSPCRKSRVFSGCVSADTLEPPSSAKVTETKGASPAFLRAGQPRLVPGETLEKSLGPGKTTAEPQHQSPPGISSEGFPWDGFNEQTPKDLPNRDGGAWVLGYRAGPACPFLLHEEREKSNRSELYLDLHPDHSLTEQDDRTPGRLQAVWPPPKTKDTEEKVGLKYTEAEYQAAILHLKREHKEEIENLQAQFELRAFHIRGEHAMITARLEETIENLKHELEHRWRGGCEERKDVCISTDDDCPPKTFRNVCVQTDRETFLKPCESESKTTRSNQLVPKKLNISSLSQLSPPNDHKDIHAALQPMEGMASNQQKALPPPPASIPPPPPLPSGLGSLSPAPPMPPVSAGPPLPPPPPPPPPLPPPSSAGPPPPPPPPPLPNSPAPPNPGGPPPAPPPPGLAPPPPPGLFFGLGSSSSQCPRKPAIEPSCPMKPLYWTRIQISDRSQNATPTLWDSLEEPDIRDPSEFEYLFSKDTTQQKKKPLSETYEKKNKVKKIIKLLDGKRSQTVGILISSLHLEMKDIQQAIFNVDDSVVDLETLAALYENRAQEDELVKIRKYYETSKEEELKLLDKPEQFLHELAQIPNFAERAQCIIFRSVFSEGITSLHRKVEIITRASKDLLHVKSVKDILALILAFGNYMNGGNRTRGQADGYSLEILPKLKDVKSRDNGINLVDYVVKYYLRYYDQEAGTEKSVFPLPEPQDFFLASQVKFEDLIKDLRKLKRQLEASEKQMVVVCKESPKEYLQPFKDKLEEFFQKAKKEHKMEESHLENAQKSFETTVRYFGMKPKSGEKEITPSYVFMVWYEFCSDFKTIWKRESKNISKERLKMAQESVSKLTSEKKVETKKINPTASLKERLRQKEASVTTN

SEQ ID NO: 5 - protein, 1726 aa, Artificial Sequence
  Synthetic
MGNQDGKLKRSAGDALHEGGGGAEDALGPRDVEATKKGSGGKKALGKHGKGGGGGGGGGESGKKKSKSDSRASVFSNLRIRKNLSKGKGAGGSREDVLDSQALQTGELDSAHSLLTKTPDLSLSADEAGLSDTECADPFEVTGPGGPGPAEARVGGRPIAEDVETAAGAQDGQRTSSGSDTDIYSFHSATEQEDLLSDIQQAIRLQQQQQQQLQLQLQQQQQQQQLQGAEEPAAPPTAVSPQPGAFLGLDRFLLGPSGGAGEAPGSPDTEQALSALSDLPESLAAEPREPQQPPSPGGLPVSEAPSLPAAQPAAKDSPSSTAFPFPEAGPGEEAAGAPVRGAGDTDEEGEEDAFEDAPRGSPGEEWAPEVGEDAPQRLGEEPEEEAQGPDAPAAASLPGSPAPSQRCFKPYPLITPCYIKTTTRQLSSPNHSPSQSPNQSPRIKRRPEPSLSRGSRTALASVAAPAKKHRADGGLAAGLSRSADWTEELGARTPRVGGSAHLLERGVASDSGGGVSPALAAKASGAPAAADGFQNVFTGRTLLEKLFSQQENGPPEEAEKFCSRIIAMGLLLPFSDCFREPCNQNAQTNAASFDQDQLYTWAAVSQPTHSLDYSEGQFPRRVPSMGPPSKPPDEEHRLEDAETEDDGESQSAVSETPQKRSDAVQKEVVDMKSEGQATVIQQLEQTIEDLRTKIAELERQYPALDTEVASGHQGLENGVTASGDVCLEALRLEEKEVRHHRILEAKSIQTSPTEEGGVLTLPPVDGLPGRPPCPPGAESGPQTKFCSEISLIVSPRRISVQLDSHQPTQSISQPPPPPSLLWSAGQGQPGSQPPHSISTEFQTSHEHSVSSAFKNSCNIPSPPPLPCTESSSSMPGLGMVPPPPPPLPGMTVPTLPSTAIPQPPPLQGTEMLPPPPPPLPGAGIPPPPPLPGAGILPLPPLPGAGIPPPPPLPGAAIPPPPPLPGAGIPLPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGVGIPPPPPLPGAGIPPPPPLPGAGIPPPPPLPGAGIPPPPPLPRVGIPPPPPLPGAGIPPPPPLPGAGIPPPPPLPGVGIPPPPPLPGVGIPPPPPLPGAGIPPPPPLPGMGIPPAPAPPLPPPGTGIPPPPLLPVSGPPLLPQVGSSTLPTPQVCGFLPPPLPSGLFGLGMNQDKGSRKQPIEPCRPMKPLYWTRIQLHSKRDSSTSLIWEKIEEPSIDCHEFEELFSKTAVKERKKPISDTISKTKAKQVVKLLSNKRSQAVGILMSSLHLDMKDIQHAVVNLDNSVVDLETLQALYENRAQSDELEKIEKHGRSSKDKENAKSLDKPEQFLYELSLIPNFSERVFCILFQSTFSESICSIRRKLELLQKLCETLKNGPGVMQVLGLVLAFGNYMNGGNKTRGQADGFGLDILPKLKDVKSSDNSRSLLSYIVSYYLRNFDEDAGKEQCLFPLPEPQDLFQASQMKFEDFQKDLRKLKKDLKACEVEAGKVYQVSSKEHMQPFKENMEQFIIQAKIDQEAEENSLTETHKCFLETTAYFFMKPKLGEKEVSPNAFFSIWHEFSSDFKDFWKKENKLLLQERVKEAEEVCRQKKGKSLYKIKPRHDSGIKAKISMKT

SEQ ID NO: 6 - protein, 166 aa, Artificial Sequence
  Synthetic
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 7 - protein, 351 aa, Artificial Sequence
  Synthetic
MALPFQKELEKYKNIDEDELLGKLSEEELKQLENVLDDLDPESAMLPAGFRQKDQTQKAATGPFDREHLLMYLEKEALEQKDREDFVPFTGEKKGRVFIPKEKPIETRKEEKVTLDPELEEALASASDTELYDLAAVLGVHNLLNNPKFDEETANNKGGKGPVRNVVKGEKVKPVFEEPPNPTNVEISLQQMKANDPSLQEVNLNNIKNIPIPTLREFAKALETNTHVKKFSLAATRSNDPVAIAFADMLKVNKTLTSLNIESNFITGTGILALVEALKENDTLTEIKIDNQRQQLGTAVEMEIAQMLEENSRILKFGYQFTKQGPRTRVAAAITKNNDLVRKKRVEADRR

SEQ ID NO: 8 - protein, 2639 aa, Artificial Sequence
  Synthetic
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 9 - protein, 629 aa, Artificial Sequence
  Synthetic
MENSTTTISREELEELQEAFNKIDIDNSGYVSDYELQDLFKEASLPLPGYKVREIVEKILSVADSNKDGKISFEEFVSLMQELKSKDISKTFRKIINKREGITAIGGTSTISSEGTQHSYSEEEKVAFVNWINKALENDPDCKHLIPMNPNDDSLFKSLADGILLCKMINLSEPDTIDERAINKKKLTPFTISENLNLALNSASAIGCTVVNIGASDLKEGKPHLVLGLLWQIIKVGLFADIEISRNEALIALLNEGEELEELMKLSPEELLLRWVNYHLTNAGWHTISNFSQDIKDSRAYFHLLNQIAPKGGEDGPAIAIDLSGINETNDLKRAGLMLQEADKLGCKQFVTPADVVSGNPKLNLAFVANLFNTYPCLHKPNNNDIDMNLLEGESKEERTFRNWMNSLGVNPYINHLYSDLADALVIFQLYEMIRVPVNWSHVNKPPYPALGGNMKKIENCNYAVELGKNKAKFSLVGIAGQDLNEGNSTLTLALVWQLMRRYTLNVLSDLGEGEKVNDEIIIKWVNQTLKSANKKTSISSFKDKSISTSLPVLDLIDAIAPNAVRQEMIRRENLSDEDKLNNAKYAISVARKIGARIYALPDDLVEVKPKMVMTVFACLMGKGLNRIK

SEQ ID NO: 10 - protein, 140 aa, Artificial Sequence
  Synthetic
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 11 - protein, 876 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRMASLSAVVVAPSVSSSAAVPPAPPLPGDSGTVIPPPPPPPPLPGGVVPPSPPLPPGTCIPPPPPLPGGACIPPPPQLPGSAAIPPPPPLPGVASIPPPPPLPGATAIPPPPPLPGATAIPPPPPLPGGTGIPPPPPPLPGSVGVPPPPPLPGGPGLPPPPPPFPGAPGIPPPPPGMGVPPPPPFGFGVPAAPVLPFGLTPKKVYKPEVQLRRPNWSKFVAEDLSQDCFWTKVKEDRFENNELFAKLTLAFSAQTKTSKAKKDQEGGEEKKSVQKKKVKELKVLDSKTAQNLSIFLGSFRMPYQEIKNVILEVNEAVLTESMIQNLIKQMPEPEQLKMLSELKEEYDDLAESEQFGVVMGTVPRLRPRLNAILFKLQFSEQVENIKPEIVSVTAACEELRKSENFSSLLELTLLVGNYMNAGSRNAGAFGFNISFLCKLRDTKSADQKMTLLHFLAELCENDHPEVLKFPDELAHVEKASRVSAENLQKSLDQMKKQIADVERDVQNFPAATDEKDKFVEKMTSFVKDAQEQYNKLRMMHSNMETLYKELGDYFVFDPKKLSVEEFFMDLHNFRNMFLQAVKENQKRRETEEKMRRAKLAKEKAEKERLEKQQKREQLIDMNAEGDETGVMD

SEQ ID NO: 12 - DNA, 6 bp, Artificial Sequence
  Synthetic
  repeat_unit (1)..(6): region may repeat 80 or more times
ggggcc

SEQ ID NO: 13 - DNA, 16 bp, Artificial Sequence
  Synthetic
ccccggccccggcccc

SEQ ID NO: 14 - DNA, 25 bp, Artificial Sequence
  Synthetic
gcttaatgccagaccgggagttgga

SEQ ID NO: 15 - DNA, 26 bp, Artificial Sequence
  Synthetic
tcatcttcatccaggtcatataaatt

SEQ ID NO: 16 - DNA, 43 bp, Artificial Sequence
  Synthetic
ggtggctctggaggcggatccgccgggtggaacgcctacatcg

SEQ ID NO: 17 - DNA, 43 bp, Artificial Sequence
  Synthetic
ttatctagatccggtggatcctcagtactgggaacgccgaagg

SEQ ID NO: 18 - DNA, 31 bp, Artificial Sequence
  Synthetic
gagactcgagccatggcttctctctctgctg

SEQ ID NO: 19 - DNA, 28 bp, Artificial Sequence
  Synthetic
gagaggatccttagcttgcacggccaac

SEQ ID NO: 20 - DNA, 20 bp, Artificial Sequence
  Synthetic
gggggacctagagagaagaa

SEQ ID NO: 21 - DNA, 20 bp, Artificial Sequence
  Synthetic
cgtagccgaacgaactcatg

SEQ ID NO: 22 - DNA, 20 bp, Artificial Sequence
  Synthetic
cgcttgtaggaggtcgaaaa

SEQ ID NO: 23 - DNA, 20 bp, Artificial Sequence
  Synthetic
agtagctggagtacgcggag

SEQ ID NO: 24 - DNA, 20 bp, Artificial Sequence
  Synthetic
acggacagcgaggaggagac

SEQ ID NO: 25 - DNA, 20 bp, Artificial Sequence
  Synthetic
atcaaagagccagagctgga

SEQ ID NO: 26 - DNA, 20 bp, Artificial Sequence
  Synthetic
ctcagatcgagattctccag

SEQ ID NO: 27 - DNA, 20 bp, Artificial Sequence
  Synthetic
ggatagacttgaggtcgttg

SEQ ID NO: 28 - DNA, 20 bp, Artificial Sequence
  Synthetic
atgaagctggcgaagcgatc

SEQ ID NO: 29 - DNA, 20 bp, Artificial Sequence
  Synthetic
gaaggctcagagtgtttctg

SEQ ID NO: 30 - DNA, 20 bp, Artificial Sequence
  Synthetic
ttctcgttagtggcgtcttc

SEQ ID NO: 31 - DNA, 20 bp, Artificial Sequence
  Synthetic
tcagcacttcttcctcatag

SEQ ID NO: 32 - DNA, 20 bp, Artificial Sequence
  Synthetic
aaagctatctcgtccatcag

SEQ ID NO: 33 - DNA, 20 bp, Artificial Sequence
  Synthetic
tctgagcatactggatctga

SEQ ID NO: 34 - DNA, 20 bp, Artificial Sequence
  Synthetic
ttggaggacacgtccatctc

SEQ ID NO: 35 - DNA, 20 bp, Artificial Sequence
  Synthetic
ttgaaccactcttcggcgtt

SEQ ID NO: 36 - DNA, 20 bp, Artificial Sequence
  Synthetic
tctcggttagcacggtgaag

SEQ ID NO: 37 - DNA, 20 bp, Artificial Sequence
  Synthetic
ttcgatctccagggtcttag

SEQ ID NO: 38 - DNA, 20 bp, Artificial Sequence
  Synthetic
ctaatgtctgcattctgctt

SEQ ID NO: 39 - DNA, 20 bp, Artificial Sequence
  Synthetic
tctccagtttgttgattgtg

SEQ ID NO: 40 - DNA, 20 bp, Artificial Sequence
  Synthetic
catcttgacattgaggaggt

SEQ ID NO: 41 - DNA, 20 bp, Artificial Sequence
  Synthetic
ctgcaatctcgatgtccaag

SEQ ID NO: 42 - DNA, 20 bp, Artificial Sequence
  Synthetic
ccttccaagagttttctgta

SEQ ID NO: 43 - DNA, 20 bp, Artificial Sequence
  Synthetic
tgaaactgagcctggtctct

SEQ ID NO: 44 - DNA, 20 bp, Artificial Sequence
  Synthetic
aagacctgcgagctctgaga

SEQ ID NO: 45 - DNA, 20 bp, Artificial Sequence
  Synthetic
aagccactgtaagcagaacg

SEQ ID NO: 46 - DNA, 20 bp, Artificial Sequence
  Synthetic
gagcgagcagacatcaagta

SEQ ID NO: 47 - DNA, 20 bp, Artificial Sequence
  Synthetic
cagctttcgtagcctcaatg

SEQ ID NO: 48 - DNA, 20 bp, Artificial Sequence
  Synthetic
ttgggaatagggctcaatct

SEQ ID NO: 49 - DNA, 20 bp, Artificial Sequence
  Synthetic
attggggagaacttttcctg

SEQ ID NO: 50 - DNA, 20 bp, Artificial Sequence
  Synthetic
tgtataggatctggaactca

SEQ ID NO: 51 - DNA, 20 bp, Artificial Sequence
  Synthetic
cctaagtcatctcagaatta

SEQ ID NO: 52 - DNA, 20 bp, Artificial Sequence
  Synthetic
tagcacaacattgaaagtcc

SEQ ID NO: 53 - DNA, 20 bp, Artificial Sequence
  Synthetic
gatactctgcgtaaggagga

SEQ ID NO: 54 - DNA, 20 bp, Artificial Sequence
  Synthetic
aaagccactctgcaagcaaa

SEQ ID NO: 55 - DNA, 20 bp, Artificial Sequence
  Synthetic
ataagcatggaccatgcaca

(19) United States
(12) Patent Application Publication
(10) Pub. No.: US 2022/0280603 A1
(43) Pub. Date: Sep. 8, 2022

(54) TARGETING OF THE CYTOSKELETON AS A THERAPEUTIC APPROACH FOR NEURODEGENERATIVE DISEASE

(71) Applicant: University of Massachusetts, Boston, MA (US)

(72) Inventors: Claudia Fallini, North Kingstown, RI (US); John Landers, Framingham, MA (US)

(73) Assignee: University of Massachusetts, Boston, MA (US)

(21) Appl. No.: 17/635,047

(22) PCT Filed: Aug. 14, 2020
(86) PCT No.: PCT/US20/46346

§ 371 Date: Feb. 14, 2022

Related U.S. Application Data
(60) Provisional application No. 62/887,439, filed on Aug. 15, 2019.

(51) Int. Cl. A61K 38/17; A61K 38/43; C07K 14/47

(52) U.S. Cl. CPC A61K 38/1709; A61K 38/43; C07K 14/47

(57) ABSTRACT

In some aspects, the disclosure relates to compositions and methods useful for the modulating the function of the nuclear pore and/or nucleocytoplasmic transport (NCT). In some embodiments, the disclosure relates to methods of treatment of a neurodegenerative disease (e.g., amyotrophic lateral sclerosis).

### RELATED APPLICATION

This application is a national stage filing under 35 U.S.C. § 371 of international PCT application PCT/US2020/046346, filed Aug. 14, 2020, which claims priority under 35 U.S.C. § 119(e) to U.S. provisional patent application, U.S. Ser. No. 62/887,439, filed Aug. 15, 2019, the entire contents of each of which are incorporated herein by reference.

### BACKGROUND

Neurodegenerative diseases such as amyotrophic lateral sclerosis (ALS) are characterized by progressive loss of motor neurons (MNs). Most ALS cases are sporadic and ˜10% are familial, yet the two classes are clinically indistinguishable suggesting that similar pathways may be responsible for the MN degeneration. Defects in nucleocytoplasmic transport (NCT) have been observed in both cellular and in vivo models of ALS and reinforced by pathological evidence in familial and sporadic ALS patients. Nuclear deficiency of RNA-binding proteins (RBPs) such as TDP-43 and FUS is a pathological hallmark of ALS, strongly supporting a link between NCT and disease pathogenesis.

NCT is a tightly regulated process that actively controls the separation and exchange between cytoplasmic and nucleoplasmic proteins and RNAs. It is centered on the function of the nuclear pore complex (NPC), a multiprotein complex spanning the whole nuclear envelope and comprised of about 30 different nucleoporins. Other key players controlling NCT are the small GTPase Ran, its GTPase-activating protein RanGAP1, and the carrier proteins importins and exportins. The cellular distribution of these factors confers directionality to the transport, while the structural integrity and density of the NPCs across the nuclear envelope modulate the efficiency of the NCT. Interestingly, some of the nucleoporins are the longest-lived proteins in the cell, and they are not replaced or replaced extremely slowly once the NPC is formed in postmitotic neurons.

### SUMMARY

The disclosure relates, in some aspects, to compositions and methods useful for the modulation of nucleocytoplasmic transport (e.g., for treatment of a disease associated with a nucleocytoplasmic transport (NCT) defect, such as Amyotrophic lateral sclerosis).

In some aspects, the disclosure provides a method of modulating the function of the nuclear pore in a cell. In some embodiments, the disclosure provides a method of modulating nucleocytoplasmic transport (NCT) in a cell. In some embodiments, the method comprises delivering a molecular agent that stabilizes the cytoskeleton to the cell. In some embodiments, the method comprises delivering a molecular agent that promotes actin and/or tubulin polymerization. In some embodiments, the method comprises delivering a molecular agent that inhibits actin and/or tubulin depolymerization.

In some aspects, the disclosure provides a method of increasing actin polymerization in a cell, the method comprising delivering a nucleic acid comprising a transgene that encodes a formin, wherein the formin comprises an FH1 domain and FH2 domain.

In some aspects, the disclosure provides a method of treating a subject having a neurodegenerative disease. In some embodiments, neurodegenerative disease is a disease associated with a nucleocytoplasmic transport (NCT) defect. In some embodiments, the method comprises administering a molecular agent that stabilizes the cytoskeleton to the subject. In some embodiments, the method comprises administering a molecular agent that promotes actin and/or tubulin polymerization to the subject. In some embodiments, the method comprises administering a molecular agent that inhibits actin and/or tubulin depolymerization to the subject. A neurodegenerative disease may be Amyotrophic lateral sclerosis (ALS), Alzheimer's disease, Huntington's disease, or Frontotemporal dementia (FTD). In some embodiments, a subject having ALS has sporadic ALS or familial ALS.

In some embodiments, a subject has a mutation in at least one gene or protein selected from the group consisting of: C9ORF72, PFN1, TUBA4A, KIF5A TDP 43, SOD1, kinesin, and Tau. A mutation in PFN1 protein may be C71G, M114T, G118V, A20T, T109M, Q139L, or E117G. A mutation in C9ORF72 may be a repeat expansion (e.g., comprising 80 or more GGGGCC repeats ((G₄C₂)₈₀)) (SEQ ID NO: 12).

In some embodiments, a cell is a neural cell (e.g., a neuroblast, a neural glial cell or a neuron). In some embodiments, a cell is a motor neuron.

In some embodiments, the method rescues actin polymerization, cytoskeletal growth, and/or division in the cell. In some embodiments, the method rescues axon growth in a neural cell.

In some embodiments, the cell comprises a PFN1 mutation or a repeat expansion in C9ORF72. A PFN1 mutation may comprise C71G, M114T, G118V, A20T, T109M, Q139L, or E117G. A repeat expansion in C9ORF72 may comprise 80 GGGGCC repeats ((G₄C₂)₈₀) (SEQ ID NO: 12).

In some embodiments, a molecular agent is a transgene (e.g., transgene encodes a protein), protein, or a small molecule. In some embodiments, the protein is an enzyme that polymerizes actin, an actin-severing protein, an actin capping protein, or an actin bundling protein.

An enzyme that polymerizes actin may be a formin, a profilin-1 (PFN1), a profilin-2 (PFN2), an Arp2/3 complex, an Ena/VASP homology protein, or a Wiskott-Aldrich syndrome protein. In some embodiments, a formin is a constitutively active formin. In some embodiments, a formin minimally comprises an FH1 domain and an FH2 domain.

An actin-severing protein may be a cofilin or a variant thereof. An actin capping protein may be a tropomodulin or a variant thereof. An actin bundling protein may be a filamin, a fimbrin or a variant thereof.

In some embodiments, a transgene is delivered using a viral vector, an antibody-drug conjugate (ADC), closed ended DNA (ceDNA), or messenger RNA (mRNA). An rAAV may comprise a capsid protein (e.g., AAV9 serotype); and a nucleic acid comprising a promoter operably linked to the transgene. In some embodiments, the delivery results in expression of the transgene in the cell.

A small molecule of the disclosure may be IMM-01, paclitaxel, swinholide, jasplakinolide, or phalloidin.

In some embodiments, modulating the function of the nuclear pore (e.g., modulating nucleocytoplasmic transport) comprises modulating the activity, expression, or localization of a nucleoporin of the FG-Nup family, Nup358/RanBP2, POM121, RanGAP1, an importin, an exportin, and/or a RNA-binding protein. A nucleoporin of the FG-Nup family may be Nup62, Nup153, Nup214, or Nup358. An importin may be Importin-β. In some embodiments, an exportin is XPO1. In some embodiments, the RNA-binding protein is TDP-43, FUS, SMN, or FMRP.

In some embodiments, administering a molecular agent to a subject that stabilizes the cytoskeleton leads to proper regulation of nucleocytoplasmic transport. In some embodiments, administering a molecular agent to a subject that stabilizes the cytoskeleton leads to increased transport of proteins and/or nucleic acids across the nuclear membrane, optionally wherein increased transport is increased nuclear import. In some embodiments, administering a molecular agent to a subject that stabilizes the cytoskeleton leads to decreased nuclear export.

In some embodiments, a transgene is administered to a subject via injection (e.g., intravenous injection, intravascular injection or intraventricular injection). In some embodiments, the administration results in expression of the transgene in the central nervous system tissue and/or the peripheral tissue of the subject.

### BRIEF DESCRIPTION OF DRAWINGS

FIGS. 1A-1H show that mutant PFN1 alters the composition and density of the nuclear pore complex (NPC).

FIGS. 1A-1C show that the localization of FG-nucleoporins (FG-Nups) the nuclear envelope pore membrane protein POM121, and Ran GTPase-activating protein 1 (RanGAP1), respectively, to the nuclear envelope (identified based on DAPI staining) is altered in a higher percentage of motor neurons (MNs) expressing V5-tagged mutant PFN1 (C71G or G118V) compared to WT PFN1 control. DAPI was used to detect the nucleus and assess cell health. *p<0.05, **p<0.01, ***p<0.001. N=3-5 independent experiments.

FIG. 1D shows that the relative expression of RAs-related Nuclear protein (Ran) in the cytoplasm compared to the nucleus (C:N ratio) is increased in MNs expressing mutant PFN1, regardless of the presence of aggregates, indicating possible functional defects in the segregation of cytoplasmic and nuclear proteins. DAPI was used to detect the nucleus and assess cell health. 76-103 cells from 6 independent experiments.

FIG. 1E shows that C71G PFN1-positive cytoplasmic inclusions are not positive for FG-Nups, POM121, RanGAP1, or Ran, suggesting no co-aggregation under these conditions. DAPI was used to detect the nucleus and assess cell health.

FIG. 1F shows that there is no difference in the solubility of Ran (middle panel) or RanGAP1 (top panel) caused by the expression of PFN1 mutants when assayed in HEK293 cells using detergent-based cellular fractionation. Triton X-100 (2%) and urea (8M) were used to extract the soluble and insoluble fraction, respectively.

FIG. 1G shows a representative co-immunoprecipitation (co-IP) assay between V5-tagged WT PFN1 or mutant PFN1; and RanGAP1 (top panel) or Ran (middle panel). No bands were detected in the IP pellet, suggesting a lack of interaction.

FIG. 1H shows a representative western blot and quantification showing unchanged levels of SUMO1-modified RanGAP1 in the presence of mutant PFN1. Antibodies against SUMO1 (top panel) and RanGAP1 (second panel) detect a band ˜80 KDa corresponding to SUMOylated RanGAP1. V5 antibody (bottom panel) shows the expression of the V5-tagged PFN1 protein, while β-tubulin was used as a loading control. 4 independent experiments.

FIGS. 2A-2C show that nuclear membrane integrity is compromised by mutant PFN1.

FIG. 2A show that transmission electron microscopy indicates the presence of protrusions and folds (arrows) in neuroblast cells (Neuro2a cells) expressing V5- or GFP-tagged C71G PFN1 compared to untransfected or WT PFN1 cells, similar to what observed in the presence of TDP-43 C-terminal fragment (CTF). Arrows point to anomalous membrane structures in the nucleus. Aggregates (asterisks) are visible as dark amorphous structures in the cytoplasm.

FIG. 2B shows that Lamin A/C distribution at the NE is altered in a higher percentage of MNs expressing mutant PFN1 compared to MNs expressing WT PFN1. DAPI was used to detect the nucleus and assess cell health. DAPI (blue) was used to detect the nucleus and assess cell health. *p<0.05, **p<0.01. N=5 independent experiments (14-47 cells)

FIG. 2C shows that the overall nuclear levels of Lamin A/C are slightly reduced in cells with abnormal staining.

FIGS. 3A-3C show that nuclear pore complex (NPC) composition is altered in patient-derived mutant PFN1 lymphoblast cells. Immunofluorescence analyses of the localization of FG-Nups (FIG. 3A), the C:N ratio of Ran (FIG. 3B), and localization of Lamin A/C and RanGAP1 (FIG. 3C) reveal alterations to the NPC composition in lymphoblast cells derived from 3 ALS patients carrying PFN1 having a C71G or G118V mutation compared to 3 control cell lines. Quantifications show an increase in the percentage of cells with disrupted Lamin A/C and RanGAP1 staining for all lines, while FG-Nups and Ran were significantly altered only in the G118V or C71G mutant lines, respectively. *p<0.05, **p<0.01, ***p<0.001, n.s. non-significant. DAPI was used to detect the nucleus and assess cell health.

FIGS. 4A-4E show that mutant PFN1 alters the efficiency of nuclear import.

FIGS. 4A-4B show time-lapse images and quantification of S-mCherry import dynamics in cortical neurons expressing GFP or GFP-tagged PFN1 and treated with Leptomycin B. S-mCherry levels are shown as 16-bit heat map. Dashed lines indicate the nucleus. *p<0.05, **p<0.01, ***p<0.001. N=44 cells from 4 independent experiments

FIGS. 4C-4D shows regression analysis of S-mCherry kinetics and the percentage of cells whose nuclear levels did not rise above 1.2 folds over initial values upon the application of Leptomycin B (i.e. non-responders). *p<0.05, **p<0.01, ***p<0.001. N=44 cells from 4 independent experiments

FIG. 4E shows that the C:N ratio of S-mCherry is not affected by mutant PFNlin untreated cells. GFP alone, used as control, localizes to both nucleus and cytoplasm. DAPI was used to detect the nucleus and assess cell health. 22-37 cells from 3 independent experiments.

FIGS. 5A-5D show that mutant PFN1 perturbs the cellular distribution and function of RNA-binding proteins (RBPs).

FIGS. 5A-5B show that mutant PFN1 causes redistribution of nuclear RBPs TDP-43 and FUS, respectively, to the cytoplasm, as quantified by their cytoplasm to nucleus (C:N) ratios. DAPI was used to detect the nucleus and assess cell health. *p<0.05, **p<0.01, ***p<0.001. N=34-47 cells from 3-4 independent experiments.

FIG. 5C shows RNA-FISH analysis demonstrating that the Nef1 mRNA expression levels in the axon, but not in the cell soma, are significantly reduced due to the expression of GFP-C71G PFN1 compared to GFP or GFP-WT PFN1. 29-53 cells from 4 independent experiments.

FIG. 5D shows a representative DNA gel and quantification of the levels of the POLDIP3 S2 variant over total POLDIP3 levels (S1+S2) in control versus mutant PFN1 lymphoblast lines. *p<0.05, **p<0.01, ***p<0.001. 4 independent experiments.

FIGS. 6A-6C show that inhibition of nuclear export rescues ALS-associated and PFN1-dependent cellular defects.

FIG. 6A shows that KPT-276 treatment rescues C:N ratio of TDP-43 in MNs expressing mutant PFN1. DAPI was used to detect the nucleus and assess cell health. DMSO was used as vehicle control. *p<0.05, ***p<0.001. 4 independent experiments.

FIG. 6B shows that MNs expressing C71G PFN1 had significantly shorter axons compared to MNs expressing WT PFN1. This defect was rescued by KPT-276 treatment. Insets show the tracing of the primary axon and the expression of V5-PFN1 in the cell body.

FIG. 6C shows representative time lapse images and quantification of axon outgrowth in the presence or absence of KPT-276. A full rescue of the outgrowth defects was observed following KPT-276 treatment. 116-207 cells from 4 independent experiments.

FIGS. 7A-7G show that actin homeostasis is a significant modulator of NPC structure and function.

FIGS. 7A-7B show that localization of RanGAP1 and Ran, respectively, is disrupted by Latrunculin A (LatA) treatment. Line plots of Ran and DAPI intensity are shown. *p<0.05, **p<0.01, ***p<0.001. N=4 independent experiments.

FIGS. 7C-7D show representative images and quantification show rescue of mislocalization of RanGAP1 and TDP-43, respectively, due to the overexpression of GFP-formin mDia1 in MNs expressing C71G PFN1. *p<0.05, **p<0.01, ***p<0.001. N=4 independent experiments.

FIGS. 7E-7G show representative images and quantification that show rescue of mislocalization of FG-Nups, Ran, and Nef1 mRNA, respectively, following treatment with IMM01 compared to DMSO control in MNs expressing C71G PFN1. 3-4 independent experiments.

FIGS. 8A-8F show that overexpression of formin mDia1 rescues nuclear import defects in mutant PFN1 neurons.

FIGS. 8A-8B show time-lapse images and quantification of S-mCherry import dynamics upon treatment with Leptomycin B in cortical neurons expressing GFP or GFP-mDia1 and V5-tagged PFN1. S-mCherry levels are shown as 16-bit heat map. Dashed lines indicate the nucleus. Cells were post-fixed and stained to verify expression of V5-PFN1 constructs. DAPI was used to detect the nucleus and assess cell health.

FIGS. 8C-8D show regression analysis of S-mCherry kinetics and percentage of non-responder cells. ***p<0.001. N=43-50 cells from 4 independent experiments.

FIG. 8E shows that overexpression of formin mDia1 rescues F-actin levels and actin-dependent morphological defects in the growth cone of MNs expressing mutant PFN1. F-actin levels were quantified in the growth cones of MNs expressing WT PFN1 or C71G PFN1. GFP-mDia1 expression restored normal F-actin polymerization and growth cone morphology in MNs expressing mutant PFN1. ***p<0.001; N=30 cells for all conditions.

FIG. 8F shows that overexpression of formin mDia1 does not alter the frequency of C71G PFN1 aggregate-containing cells. n.s.: non-significant; N=3.

FIGS. 9A-9E show that actin modulates NPC function in motor neurons (MNs) expressing a C9ORF72 mutation, a relevant ALS mutation.

FIG. 9A shows overexpression of GFP-formin mDia1 provides significant rescue of mislocalization of RanGAP1 to the nuclear envelope (NE) in MNs expressing the C9ORF72 repeat expansion (G₄C₂)₈₀ (SEQ ID NO:12). relative to overexpression of GFP alone. DAPI was used to detect the nucleus and assess cell health. *p<0.05. N=4.

FIG. 9B shows that fibroblasts derived from 3 ALS patients carrying C9ORF72 repeat expansions show increased mislocalization of both RanGAP1 and FG-Nups compared to 3 healthy controls. Treatment of these cells with IMM01 significantly rescued the defects. DMSO was used as vehicle control. DAPI was used to detect the nucleus and assess cell health. *p<0.05, ***p<0.001. N=5.

FIGS. 9C-9E show time-lapse images, quantification, and regression analysis of S-mCherry import dynamics in cortical neurons expressing GFP or GFP-formin mDia1 and C9ORF72 (G₄C₂)₈₀ (SEQ ID NO: 12). S-mCherry levels are shown as 16-bit heat map. Dashed lines indicate the nucleus. 51-64 cells from 5 independent experiments.

### DETAILED DESCRIPTION

Aspects of the disclosure relate to methods of modulating the function of the nuclear pore (e.g., modulating nucleocytoplasmic transport) in a cell. In some embodiments, methods of modulating comprise delivering a molecular agent that stabilizes the cytoskeleton (e.g., promotes polymerization of actin and/or tubulin; or inhibits actin and/or tubulin depolymerization) to the cell. Other aspects relate to methods of treating a subject having a disease associated with a nucleocytoplasmic transport (NCT) defect. In some embodiments, methods of treating comprise administering a molecular agent that stabilizes the cytoskeleton (e.g., promotes polymerization of actin and/or tubulin; or inhibits actin and/or tubulin depolymerization) to the subject.

Amyotrophic lateral sclerosis (ALS) is a fatal neurodegenerative disease of unknown etiology. Herein, the inventors show that genetic and pharmacological modulation of actin polymerization disrupts nuclear pore integrity, nuclear import, and downstream pathways such as mRNA post-transcriptional regulation. Importantly, modulation of actin homeostasis was shown by the inventors to rescue nuclear pore instability and dysfunction caused by mutant PFN1 as well as by C9ORF72 repeat expansion, the most common mutation in ALS patients.

Toxic insults such as oxidative stress and protein aggregation negatively impact NCT. Many mutant ALS-linked proteins show an increased tendency to aggregate, including SOD1, TDP-43, FUS, and Profilin1 (PFN1). PFN1 is a small actin-binding protein that positively regulates actin polymerization in a formin-dependent manner. In some embodiments, subjects (e.g., patients) have at least one mutation in PFN1 (e.g., as least one of C71G, M114T, G118V, A20T, T109M, Q139L, and/or E117G). In some embodiments, a mutation in PFN1 causes PFN1 to be unstable and prone to aggregation, leading to the formation of cytoplasmic inclusions. A mutation in PFN1 can also impair its association with filamentous (F)-actin. In some embodiments, a motor neuron (MN) expressing a mutant PFN1 (e.g., a C71G and/or G118V mutation) shows morphological abnormalities (e.g., smaller growth cones and shorter axons). In some embodiments, a mutation in PFN1 causes a cell or subject to develop neurodegenerative disease (e.g., Amyotrophic lateral sclerosis). Herein, the inventors demonstrate that mutant PFN1 disrupt the NCT and the normal function of RNA-binding proteins and motor neurons. In some embodiments, modulating actin homeostasis is capable of modulating (e.g., rescuing) NCT defects, e.g., caused by mutant PFN1 and/or C9ORF72 repeat expansions. In some embodiments, a molecular agent (e.g., formin) that stabilizes the cytoskeleton (e.g., promotes polymerization of actin and/or tubulin; or inhibits actin and/or tubulin depolymerization) is capable of modulating (e.g., rescuing) NCT defects, e.g., caused by mutant PFN1 and/or C9ORF72 repeat expansions. In some embodiments, actin modulates the integrity of the nuclear envelope (NE) via the function of the linker of nucleoskeleton and cytoskeleton (LINC) complex, a multiprotein complex that physically connects lamins with actin and microtubules. In some embodiments, mutations in LINC components cause cerebellar ataxia.

### Methods of Modulating the Function of the Nuclear Pore

Some aspects of the disclosure involve a method of modulating the function of the nuclear pore (e.g., modulating nucleocytoplasmic transport) in a cell or a subject (e.g., rescuing an NCT defect). In some embodiments, the method comprises delivering a molecular agent that stabilizes actin and/or tubulin (e.g., a protein or small molecule that stabilizes actin and/or tubulin). Other aspects of the disclosure involve a method of increasing actin polymerization in a cell or a subject. In some embodiments, the method comprises delivering a nucleic acid comprising a transgene that encodes a protein that stabilizes the cytoskeleton (e.g., promotes polymerization of actin and/or tubulin; or inhibits actin and/or tubulin depolymerization). In some embodiments, the cell is in vitro, in vivo (i.e., in a subject), or ex vivo.

In some embodiments, a method of modulating the function of the nuclear pore (e.g., modulating nucleocytoplasmic transport) described herein rescues actin polymerization, stabilizes actin polymerization, inhibits actin depolymerization, increases cytoskeletal growth, and/or increases cellular division. In some embodiments, a method of modulating the function of the nuclear pore involves the rescue of axon growth in a neural cell (e.g., a motor neuron). In some embodiments, a method of modulating the function of the nuclear pore comprises modulating the activity, expression, or localization of a gene or protein involved in the function of the nuclear pore and/or NCT. In some embodiments, a gene or protein involved in the function of the nuclear pore and/or NCT is a member of the FG-nucleoporin (FG-Nup) family, RAN binding protein 2 (RANBP2) (also known as nucleoporin 358 (Nup358)), nuclear envelope pore membrane protein POM 121, Ran GTPase-activating protein 1 (RanGAP1), an importin (e.g., importin-α and importin-β), an exportin (e.g., exportin-1 (XPO1)), and/or a RNA-binding protein (RBP).

In some embodiments, a member of the FG-Nup family is Nup62, Nup153, Nup214, or Nup358. In some embodiments, the RNA-binding protein (RBP) is TDP-43, FUS, SMN, or FMRP. In some embodiments, a method of modulating the function of the nuclear pore (e.g., modulating nucleocytoplasmic transport) corrects cytosolic mislocalization of a nuclear RBP to increase the localization of a RBP to the nucleus.

In some embodiments, a method of modulating the function of the nuclear pore and/or NCT leads to increased transport of proteins and/or nucleic acids across the nuclear membrane. In some embodiments, increased transport of proteins and/or nucleic acids across the nuclear membrane involves increased nuclear import (e.g., increased transport of protein and/or nucleic acids from the cytoplasm into the nucleus). In some embodiments, modulating the function of the nuclear pore and/or NCT leads to decreased nuclear export (e.g., decreased transport of protein and/or nucleic acids from the nucleus into the cytoplasm).

### Molecular Agent

In some aspects, the disclosure provides a molecular agent that promotes actin polymerization or inhibits actin depolymerization. A molecular agent may be a transgene (e.g., a transgene that encodes a protein), protein, or a small molecule. In some embodiments, a transgene is delivered using a viral vector, an antibody-drug conjugate (ADC), closed ended DNA (ceDNA), or messenger RNA (mRNA). In some embodiments, a transgene is delivered using a recombinant adeno-associated virus (rAAV). A molecular agent that promotes actin polymerization (e.g., an enzyme that promotes actin polymerization) or inhibits actin depolymerization further promotes actin severing (e.g., an actin-severing protein), actin capping (e.g., an actin capping protein), and/or actin bundling (e.g., an actin bundling protein).

In some embodiments, an enzyme that polymerizes actin is a formin, a profilin-1 (PFN1), a profilin-2 (PFN2), an Arp2/3 complex, an Ena/VASP homology protein, or a Wiskott-Aldrich syndrome protein.

In some embodiments, a formin is a formin 1 or formin 2 protein. A formin is involved in actin polymerization and may associate with the fast-growing end of an actin filament. In some embodiments, a formin is a formin 1 protein is as provided by NCBI Gene ID: 342184. In some embodiments, a formin is a formin 2 protein is as provided by NCBI Gene ID: 56776. A formin may be a constitutively active formin (e.g., a formin that continually functions to polymerize actin). In some embodiments, a constitutively active formin has an enzymatic activity that is 50%, 60%, 70%, 80%, 90%, 100%, 110%, or 120% as active as a wild-type or control formin protein. In some embodiments, a formin comprises a FH1 domain, a FH2 domain, and a FH3 domain. In some embodiments, a formin comprises a FH1 domain and a FH3 domain. In some embodiments, a formin comprises a FH2 domain and a FH3 domain. In some embodiments, a FH1 domain comprises an amino acid sequence as provided by SEQ ID NO: 1. In some embodiments, a FH1 domain comprises an amino acid sequence that is at least 70%, 75%, 80%, 85%, 90%, or 95% identical to SEQ ID NO: 1. In some embodiments, a FH2 domain comprises an amino acid sequence as provided by SEQ ID NO: 2. In some embodiments, a FH2 domain comprises an amino acid sequence that is at least 70%, 75%, 80%, 85%, 90%, or 95% identical to SEQ ID NO: 2. In some embodiments, a formin comprising a FH1 domain and a FH2 domain comprises an amino acid sequence as provided by SEQ ID NO: 3. In some embodiments, a formin comprising a FH1 domain and a FH2 domain comprises an amino acid sequence that is at least 70%, 75%, 80%, 85%, 90%, or 95% identical to SEQ ID NO: 3. In some embodiments, a formin comprises an amino acid sequence as provided by SEQ ID NO: 4 or SEQ ID NO: 5. In some embodiments, a formin comprises an amino acid sequence that is at least 70%, 75%, 80%, 85%, 90%, or 95% identical to SEQ ID NO: 4 or SEQ ID NO: 5.

FH1 domain of a formin
(SEQ ID NO: 1)
MASLSAVVVAPSVSSSAAVPPAPPLPGDSGTVIPPPPPPPPLPGGVVPPSPPLPPGTCIPPPP
PLPGGACIPPPPQLPGSAAIPPPPPLPGVASIPPPPPLPGATAIPPPPPLPGATAIPPPPPLP
GGTGIPPPPPPLPGSVGVPPPPPLPGGPGLPPPPPPFPGAPGIPPPPPGMGVPPPPPFGFGVP
AAPVLPFGLTP
FH2 domain of a formin
(SEQ ID NO: 2)
KKVYKPEVQLRRPNWSKFVAEDLSQDCFWTKVKEDRFENNELFAKLTLAFSAQTKTSKAKKDQ
EGGEEKKSVQKKKVKELKVLDSKTAQNLSIFLGSFRMPYQEIKNVILEVNEAVLTESMIQNLI
KQMPEPEQLKMLSELKEEYDDLAESEQFGVVMGTVPRLRPRLNAILFKLQFSEQVENIKPEIV
SVTAACEELRKSENFSSLLELTLLVGNYMNAGSRNAGAFGFNISFLCKLRDTKSADQKMTLLH
FLAELCENDHPEVLKFPDELAHVEKASRVSAENLQKSLDQMKKQIADVERDVQNFPAATDEKD
KFVEKMTSFVKDAQEQYNKLRMMHSNMETLYKELGDYFVFDPKKLSVEEFFMDLHNFRNMFLQ
AVKENQKRRETEEKMRRAKLAKEKAEKERLEKQQKREQLIDMNAEGDETGVMD
FH1-FH2 domains of a formin
(SEQ ID NO: 3)
MASLSAVVVAPSVSSSAAVPPAPPLPGDSGTVIPPPPPPPPLPGGVVPPSPPLPPGTCIPPPP
PLPGGACIPPPPQLPGSAAIPPPPPLPGVASIPPPPPLPGATAIPPPPPLPGATAIPPPPPLP
GGTGIPPPPPPLPGSVGVPPPPPLPGGPGLPPPPPPFPGAPGIPPPPPGMGVPPPPPFGFGVP
AAPVLPFGLTPKKVYKPEVQLRRPNWSKFVAEDLSQDCFWTKVKEDRFENNELFAKLTLAFSA
QTKTSKAKKDQEGGEEKKSVQKKKVKELKVLDSKTAQNLSIFLGSFRMPYQEIKNVILEVNEA
VLTESMIQNLIKQMPEPEQLKMLSELKEEYDDLAESEQFGVVMGTVPRLRPRLNAILFKLQFS
EQVENIKPEIVSVTAACEELRKSENFSSLLELTLLVGNYMNAGSRNAGAFGFNISFLCKLRDT
KSADQKMTLLHFLAELCENDHPEVLKFPDELAHVEKASRVSAENLQKSLDQMKKQIADVERDV
QNFPAATDEKDKFVEKMTSFVKDAQEQYNKLRMMHSNMETLYKELGDYFVFDPKKLSVEEFFM
DLHNFRNMFLQAVKENQKRRETEEKMRRAKLAKEKAEKERLEKQQKREQLIDMNAEGDETGVM
D
Formin-1 (NCBI Sequence: NP_001264242.1; human)
(SEQ ID NO: 4)
MEGTHCTLQLHKPITELCYISFCLPKGEVRGFSYKGTVTLDRSNKGFHNCYQVREESDIISLS
QEPDEHPGDIFFKQTPTKDILTELYKLTTERERLLTNLLSSDHILGITMGNQEGKLQELSVSL
APEDDCFQSAGDWQGELPVGPLNKRSTHGNKKPRRSSGRRESFGALPQKRTKRKGRGGRESAP
LMGKDKICSSHSLPLSRTRPNLWVLEEKGNLLPNGALACSLQRRESCPPDIPKTPDTDLGFGS
FETAFKDTGLGREVLPPDCSSTEAGGDGIRRPPSGLEHQQTGLSESHQDPEKHPEAEKDEMEK
PAKRTCKQKPVSKVVAKVQDLSSQVQRVVKTHSKGKETIAIRPAAHAEFVPKADLLTLPGAEA
GAHGSRRQGKERQGDRSSQSPAGETASISSVSASAEGAVNKVPLKVIESEKLDEAPEGKRLGF
PVHTSVPHTRPETRNKRRAGLPLGGHKSLFLDLPHKVGPDSSQPRGDKKKPSPPAPAALGKVF
NNSASQSSTHKQTSPVPSPLSPRLPSPQQHHRILRLPALPGEREAALNDSPCRKSRVFSGCVS
ADTLEPPSSAKVTETKGASPAFLRAGQPRLVPGETLEKSLGPGKTTAEPQHQSPPGISSEGFP
WDGFNEQTPKDLPNRDGGAWVLGYRAGPACPFLLHEEREKSNRSELYLDLHPDHSLTEQDDRT
PGRLQAVWPPPKTKDTEEKVGLKYTEAEYQAAILHLKREHKEEIENLQAQFELRAFHIRGEHA
MITARLEETIENLKHELEHRWRGGCEERKDVCISTDDDCPPKTFRNVCVQTDRETFLKPCESE
SKTTRSNQLVPKKLNISSLSQLSPPNDHKDIHAALQPMEGMASNQQKALPPPPASIPPPPPLP
SGLGSLSPAPPMPPVSAGPPLPPPPPPPPPLPPPSSAGPPPPPPPPPLPNSPAPPNPGGPPPA
PPPPGLAPPPPPGLFFGLGSSSSQCPRKPAIEPSCPMKPLYWTRIQISDRSQNATPTLWDSLE
EPDIRDPSEFEYLFSKDTTQQKKKPLSETYEKKNKVKKIIKLLDGKRSQTVGILISSLHLEMK
DIQQAIFNVDDSVVDLETLAALYENRAQEDELVKIRKYYETSKEEELKLLDKPEQFLHELAQI
PNFAERAQCIIFRSVFSEGITSLHRKVEIITRASKDLLHVKSVKDILALILAFGNYMNGGNRT
RGQADGYSLEILPKLKDVKSRDNGINLVDYVVKYYLRYYDQEAGTEKSVFPLPEPQDFFLASQ
VKFEDLIKDLRKLKRQLEASEKQMVVVCKESPKEYLQPFKDKLEEFFQKAKKEHKMEESHLEN
AQKSFETTVRYFGMKPKSGEKEITPSYVFMVWYEFCSDFKTIWKRESKNISKERLKMAQESVS
KLTSEKKVETKKINPTASLKERLRQKEASVTTN
Formin-2 (NCBI Sequence: NP_001292353.1; human)
(SEQ ID NO: 5)
MGNQDGKLKRSAGDALHEGGGGAEDALGPRDVEATKKGSGGKKALGKHGKGGGGGGGGGESGK
KKSKSDSRASVFSNLRIRKNLSKGKGAGGSREDVLDSQALQTGELDSAHSLLTKTPDLSLSAD
EAGLSDTECADPFEVTGPGGPGPAEARVGGRPIAEDVETAAGAQDGQRTSSGSDTDIYSFHSA
TEQEDLLSDIQQAIRLQQQQQQQLQLQLQQQQQQQQLQGAEEPAAPPTAVSPQPGAFLGLDRF
LLGPSGGAGEAPGSPDTEQALSALSDLPESLAAEPREPQQPPSPGGLPVSEAPSLPAAQPAAK
DSPSSTAFPFPEAGPGEEAAGAPVRGAGDTDEEGEEDAFEDAPRGSPGEEWAPEVGEDAPQRL
GEEPEEEAQGPDAPAAASLPGSPAPSQRCFKPYPLITPCYIKTTTRQLSSPNHSPSQSPNQSP
RIKRRPEPSLSRGSRTALASVAAPAKKHRADGGLAAGLSRSADWTEELGARTPRVGGSAHLLE
RGVASDSGGGVSPALAAKASGAPAAADGFQNVFTGRTLLEKLFSQQENGPPEEAEKFCSRIIA
MGLLLPFSDCFREPCNQNAQTNAASFDQDQLYTWAAVSQPTHSLDYSEGQFPRRVPSMGPPSK
PPDEEHRLEDAETEDDGESQSAVSETPQKRSDAVQKEVVDMKSEGQATVIQQLEQTIEDLRTK
IAELERQYPALDTEVASGHQGLENGVTASGDVCLEALRLEEKEVRHHRILEAKSIQTSPTEEG
GVLTLPPVDGLPGRPPCPPGAESGPQTKFCSEISLIVSPRRISVQLDSHQPTQSISQPPPPPS
LLWSAGQGQPGSQPPHSISTEFQTSHEHSVSSAFKNSCNIPSPPPLPCTESSSSMPGLGMVPP
PPPPLPGMTVPTLPSTAIPQPPPLQGTEMLPPPPPPLPGAGIPPPPPLPGAGILPLPPLPGAG
IPPPPPLPGAAIPPPPPLPGAGIPLPPPLPGAGIPPPPPLPGAGIPPPPPLPGAGIPPPPPLP
GAGIPPPPPLPGAGIPPPPPLPGAGIPPPPPLPGAGIPPPPPLPGAGIPPPPPLPGAGIPPPP
PLPGAGIPPPPPLPGAGIPPPPPLPGAGIPPPPPLPGVGIPPPPPLPGAGIPPPPPLPGAGIP
PPPPLPGAGIPPPPPLPRVGIPPPPPLPGAGIPPPPPLPGAGIPPPPPLPGVGIPPPPPLPGV
GIPPPPPLPGAGIPPPPPLPGMGIPPAPAPPLPPPGTGIPPPPLLPVSGPPLLPQVGSSTLPT
PQVCGFLPPPLPSGLFGLGMNQDKGSRKQPIEPCRPMKPLYWTRIQLHSKRDSSTSLIWEKIE
EPSIDCHEFEELFSKTAVKERKKPISDTISKTKAKQVVKLLSNKRSQAVGILMSSLHLDMKDI
QHAVVNLDNSVVDLETLQALYENRAQSDELEKIEKHGRSSKDKENAKSLDKPEQFLYELSLIP
NFSERVFCILFQSTFSESICSIRRKLELLQKLCETLKNGPGVMQVLGLVLAFGNYMNGGNKTR
GQADGFGLDILPKLKDVKSSDNSRSLLSYIVSYYLRNFDEDAGKEQCLFPLPEPQDLFQASQM
KFEDFQKDLRKLKKDLKACEVEAGKVYQVSSKEHMQPFKENMEQFIIQAKIDQEAEENSLTET
HKCFLETTAYFFMKPKLGEKEVSPNAFFSIWHEFSSDFKDFWKKENKLLLQERVKEAEEVCRQ
KKGKSLYKIKPRHDSGIKAKISMKT

In some embodiments, an actin-severing protein is a cofilin (e.g., cofilin 1, cofilin 2, or destrin). In some embodiments, a cofilin is a protein with about 70% sequence identity to ADF protein. A cofilin may bind monomeric (G-actin) and/or filamentous actin (F-actin). In some embodiments, a cofilin is a cofilin 1 protein is as provided by NCBI Gene ID: 1072. In some embodiments, a cofilin protein comprises an amino acid sequence as provided by SEQ ID NO: 6. In some embodiments, a cofilin protein comprises an amino acid sequence that is at least 70%, 75%, 80%, 85%, 90%, or 95% identical to SEQ ID NO: 6.

Cofilin 1 (NCBI Sequence: NP_055363.1; human)
(SEQ ID NO: 6)
MASGVAVSDGVIKVFNDMKVRKSSTPEEVKKRKKAVLFCLSEDKKNIILE
EGKEILVGDVGQTVDDPYATFVKMLPDKDCRYALYDATYETKESKKEDLV
FIFWAPESAPLKSKMIYASSKDAIKKKLTGIKHELQANCYEEVKDRCTLA
EKLGGSAVISLEGKPL

In some embodiments, an actin-capping protein is a tropomodulin (e.g., tropomodulin-1, tropomodulin-2, tropomodulin-3, or tropomodulin-4). A tropomodulin may bind and cap the minus end of actin. In some embodiments, a tropomodulin is a neuronal-specific tropomodulin (e.g., tropomodulin-2). In some embodiments, a tropomodulin-2 protein is as provided by NCBI Gene ID: 29767. In some embodiments, a tropomodulin-2 protein comprises an amino acid sequence as provided by SEQ ID NO: 7. In some embodiments, a tropomodulin-2 protein comprises an amino acid sequence that is at least 70%, 75%, 80%, 85%, 90%, or 95% identical to SEQ ID NO: 7.

Tropomodulin-2 (NCBI Sequence: NP_005498.1;
human)
(SEQ ID NO: 7)
MALPFQKELEKYKNIDEDELLGKLSEEELKQLENVLDDLDPESAMLPAGF
RQKDQTQKAATGPFDREHLLMYLEKEALEQKDREDFVPFTGEKKGRVFIP
KEKPIETRKEEKVTLDPELEEALASASDTELYDLAAVLGVHNLLNNPKFD
EETANNKGGKGPVRNVVKGEKVKPVFEEPPNPTNVEISLQQMKANDPSLQ
EVNLNNIKNIPIPTLREFAKALETNTHVKKFSLAATRSNDPVAIAFADML
KVNKTLTSLNIESNFITGTGILALVEALKENDTLTEIKIDNQRQQLGTAV
EMEIAQMLEENSRILKFGYQFTKQGPRTRVAAAITKNNDLVRKKRVEADR
R

In some embodiments, an actin-bundling protein is a filamin (e.g., filamin A) or a fimbrin. An actin-bundling protein may bind actin (e.g., actin filaments) and crosslink actin (e.g., actin filaments) into higher order actin structures. In some embodiments, a filamin A protein is as provided by NCBI Gene ID: 2316. In some embodiments, a filamin A protein comprises an amino acid sequence as provided by SEQ ID NO: 8. In some embodiments, a filamin A protein comprises an amino acid sequence that is at least 70%, 75%, 80%, 85%, 90%, or 95% identical to SEQ ID NO: 8. In some embodiments, a fimbrin protein is as provided by NCBI Gene ID: 5357. In some embodiments, a fimbrin protein comprises an amino acid sequence as provided by SEQ ID NO: 9. In some embodiments, a fimbrin protein comprises an amino acid sequence that is at least 70%, 75%, 80%, 85%, 90%, or 95% identical to SEQ ID NO: 9.

Filamin A (NCBI Sequence: NP_001447.2; human)
(SEQ ID NO: 8)
MSSSHSRAGQSAAGAAPGGGVDTRDAEMPATEKDLAEDAPWKKIQQNTFT
RWCNEHLKCVSKRIANLQTDLSDGLRLIALLEVLSQKKMHRKHNQRPTFR
QMQLENVSVALEFLDRESIKLVSIDSKAIVDGNLKLILGLIWTLILHYSI
SMPMWDEEEDEEAKKQTPKQRLLGWIQNKLPQLPITNFSRDWQSGRALGA
LVDSCAPGLCPDWDSWDASKPVTNAREAMQQADDWLGIPQVITPEEIVDP
NVDEHSVMTYLSQFPKAKLKPGAPLRPKLNPKKARAYGPGIEPTGNMVKK
RAEFTVETRSAGQGEVLVYVEDPAGHQEEAKVTANNDKNRTFSVWYVPEV
TGTHKVTVLFAGQHIAKSPFEVYVDKSQGDASKVTAQGPGLEPSGNIANK
TTYFEIFTAGAGTGEVEVVIQDPMGQKGTVEPQLEARGDSTYRCSYQPTM
EGVHTVHVTFAGVPIPRSPYTVTVGQACNPSACRAVGRGLQPKGVRVKET
ADFKVYTKGAGSGELKVTVKGPKGEERVKQKDLGDGVYGFEYYPMVPGTY
IVTITWGGQNIGRSPFEVKVGTECGNQKVRAWGPGLEGGVVGKSADFVVE
AIGDDVGTLGFSVEGPSQAKIECDDKGDGSCDVRYWPQEAGEYAVHVLCN
SEDIRLSPFMADIRDAPQDFHPDRVKARGPGLEKTGVAVNKPAEFTVDAK
HGGKAPLRVQVQDNEGCPVEALVKDNGNGTYSCSYVPRKPVKHTAMVSWG
GVSIPNSPFRVNVGAGSHPNKVKVYGPGVAKTGLKAHEPTYFTVDCAEAG
QGDVSIGIKCAPGVVGPAEADIDFDIIRNDNDTFTVKYTPRGAGSYTIMV
LFADQATPTSPIRVKVEPSHDASKVKAEGPGLSRTGVELGKPTHFTVNAK
AAGKGKLDVQFSGLTKGDAVRDVDIIDHHDNTYTVKYTPVQQGPVGVNVT
YGGDPIPKSPFSVAVSPSLDLSKIKVSGLGEKVDVGKDQEFTVKSKGAGG
QGKVASKIVGPSGAAVPCKVEPGLGADNSVVRFLPREEGPYEVEVTYDGV
PVPGSPFPLEAVAPTKPSKVKAFGPGLQGGSAGSPARFTIDTKGAGTGGL
GLTVEGPCEAQLECLDNGDGTCSVSYVPTEPGDYNINILFADTHIPGSPF
KAHVVPCFDASKVKCSGPGLERATAGEVGQFQVDCSSAGSAELTIEICSE
AGLPAEVYIQDHGDGTHTITYIPLCPGAYTVTIKYGGQPVPNFPSKLQVE
PAVDTSGVQCYGPGIEGQGVFREATTEFSVDARALTQTGGPHVKARVANP
SGNLTETYVQDRGDGMYKVEYTPYEEGLHSVDVTYDGSPVPSSPFQVPVT
EGCDPSRVRVHGPGIQSGTTNKPNKFTVETRGAGTGGLGLAVEGPSEAKM
SCMDNKDGSCSVEYIPYEAGTYSLNVTYGGHQVPGSPFKVPVHDVTDASK
VKCSGPGLSPGMVRANLPQSFQVDTSKAGVAPLQVKVQGPKGLVEPVDVV
DNADGTQTVNYVPSREGPYSISVLYGDEEVPRSPFKVKVLPTHDASKVKA
SGPGLNTTGVPASLPVEFTIDAKDAGEGLLAVQITDPEGKPKKTHIQDNH
DGTYTVAYVPDVTGRYTILIKYGGDEIPFSPYRVRAVPTGDASKCTVTGA
GIGPTIQIGEETVITVDTKAAGKGKVTCTVCTPDGSEVDVDVVENEDGTF
DIFYTAPQPGKYVICVRFGGEHVPNSPFQVTALAGDQPSVQPPLRSQQLA
PQYTYAQGGQQTWAPERPLVGVNGLDVTSLRPFDLVIPFTIKKGEITGEV
RMPSGKVAQPTITDNKDGTVTVRYAPSEAGLHEMDIRYDNMHIPGSPLQF
YVDYVNCGHVTAYGPGLTHGVVNKPATFTVNTKDAGEGGLSLAIEGPSKA
EISCTDNQDGTCSVSYLPVLPGDYSILVKYNEQHVPGSPFTARVTGDDSM
RMSHLKVGSAADIPINISETDLSLLTATVVPPSGREEPCLLKRLRNGHVG
ISFVPKETGEHLVHVKKNGQHVASSPIPVVISQSEIGDASRVRVSGQGLH
EGHTFEPAEFIIDTRDAGYGGLSLSIEGPSKVDINTEDLEDGTCRVTYCP
TEPGNYIINIKFADQHVPGSPFSVKVTGEGRVKESITRRRRAPSVANVGS
HCDLSLKIPEISIQDMTAQVTSPSGKTHEAEIVEGENHTYCIRFVPAEMG
THTVSVKYKGQHVPGSPFQFTVGPLGEGGAHKVRAGGPGLERAEAGVPAE
FSIWTREAGAGGLAIAVEGPSKAEISFEDRKDGSCGVAYVVQEPGDYEVS
VKFNEEHIPDSPFVVPVASPSGDARRLTVSSLQESGLKVNQPASFAVSLN
GAKGAIDAKVHSPSGALEECYVTEIDQDKYAVRFIPRENGVYLIDVKFNG
THIPGSPFKIRVGEPGHGGDPGLVSAYGAGLEGGVTGNPAEFVVNTSNAG
AGALSVTIDGPSKVKMDCQECPEGYRVTYTPMAPGSYLISIKYGGPYHIG
GSPFKAKVTGPRLVSNHSLHETSSVFVDSLTKATCAPQHGAPGPGPADAS
KVVAKGLGLSKAYVGQKSSFTVDCSKAGNNMLLVGVHGPRTPCEEILVKH
VGSRLYSVSYLLKDKGEYTLVVKWGDEHIPGSPYRVVVP
Fimbrin (NCBI Sequence: NP_001138791.1; human)
(SEQ ID NO: 9)
MENSTTTISREELEELQEAFNKIDIDNSGYVSDYELQDLFKEASLPLPGY
KVREIVEKILSVADSNKDGKISFEEFVSLMQELKSKDISKTFRKIINKRE
GITAIGGTSTISSEGTQHSYSEEEKVAFVNWINKALENDPDCKHLIPMNP
NDDSLFKSLADGILLCKMINLSEPDTIDERAINKKKLTPFTISENLNLAL
NSASAIGCTVVNIGASDLKEGKPHLVLGLLWQIIKVGLFADIEISRNEAL
IALLNEGEELEELMKLSPEELLLRWVNYHLTNAGWHTISNFSQDIKDSRA
YFHLLNQIAPKGGEDGPAIAIDLSGINETNDLKRAGLMLQEADKLGCKQF
VTPADVVSGNPKLNLAFVANLFNTYPCLHKPNNNDIDMNLLEGESKEERT
FRNWMNSLGVNPYINHLYSDLADALVIFQLYEMIRVPVNWSHVNKPPYPA
LGGNMKKIENCNYAVELGKNKAKFSLVGIAGQDLNEGNSTLTLALVWQLM
RRYTLNVLSDLGEGEKVNDEIIIKWVNQTLKSANKKTSISSFKDKSISTS
LPVLDLIDAIAPNAVRQEMIRRENLSDEDKLNNAKYAISVARKIGARIYA
LPDDLVEVKPKMVMTVFACLMGKGLNRIK

A molecular agent of the disclosure may be a small molecule (e.g., a small molecule drug). In some embodiments, a small molecule that promotes actin polymerization or inhibits actin depolymerization is IMM-01, IMM-2, paclitaxel, swinholide, jasplakinolide, or phalloidin. In some embodiments, a small molecule of the disclosure is a derivative of IMM-01, IMM-2, paclitaxel, swinholide, jasplakinolide, or phalloidin (e.g., a deuterated derivative). In some embodiments, a small molecule of the disclosure is a molecule that activates a protein that stabilizes or polymerizes actin (e.g., formin). In some embodiments, a small molecule of the disclosure is as described in Lash, L. L. et al. Small-molecule intramimics of formin autoinhibition: a new strategy to target the cytoskeletal remodeling machinery in cancer cells. Cancer Res. 2013 Nov 15; 73(22):6793-803; and Crochiere, M. L. et al. A method for quantification of exportin-1 (XPO1) occupancy by Selective Inhibitor of Nuclear Export (SINE) compounds. Oncotarget 7(2), December 2015.

TABLE 1
Examples of small molecules of the disclosure

| | |
|---|---|
| IMM-01 | 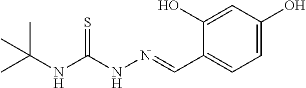 [SMILES: CC(C)(C)NC(=S)N/N=C/c1ccc(O)cc1O] |
| IMM-02 | 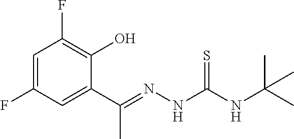 [SMILES: C/C(=N\NC(=S)NC(C)(C)C)c1cc(F)cc(F)c1O] |
| paclitaxel | 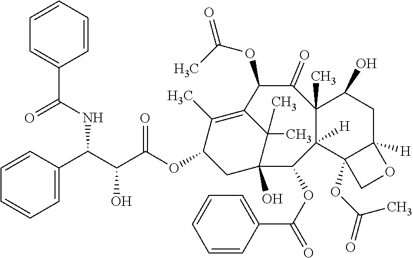 |
| swinholide | 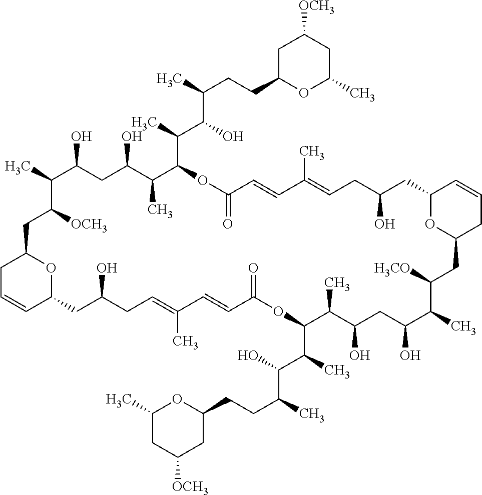 |
| jasplakinolide | 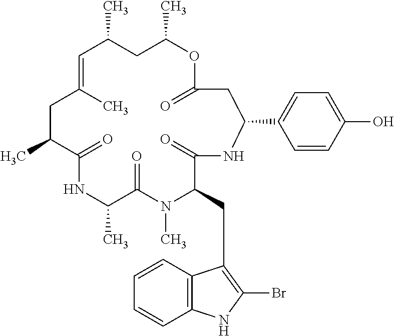 [SMILES: C/C1=C\[C@H](C)C[C@H](C)OC(=O)C[C@H](c2ccc(O)cc2)NC(=O)[C@@H](Cc2c(Br)[nH]c3ccccc23)N(C)C(=O)[C@H](C)NC(=O)[C@@H](C)C1] |
| phalloidin | 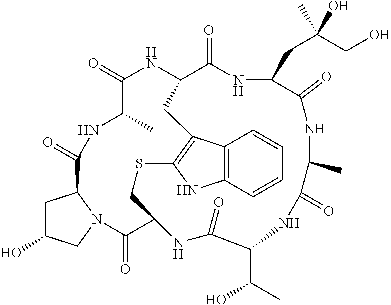 [SMILES: C[C@@H]1NC(=O)[C@H](C[C@@](C)(O)CO)NC(=O)[C@@H]2Cc3c([nH]c4ccccc34)SC[C@@H](NC(=O)[C@@H]([C@H](C)O)NC1=O)C(=O)N1C[C@H](O)C[C@H]1C(=O)N[C@@H](C)C(=O)N2] |

### Cells

A cell of the disclosure may be a human, rodent, non-human primate, or other mammalian cell. In some embodiments, a cell is a neural cell. A neural cell may be a neuroblast, a neural glial cell or a neuron (e.g., a motor neuron).

In some embodiments, a cell comprises at least one mutation that causes a defect in nucleocytoplasmic transport (NCT). In some embodiments, a mutation that causes a defect in NCT includes mutations in profilin-1 (PFN1). In some embodiments, a mutation that causes a defect in NCT includes mutations in the C9ORF72 gene. In some embodiments, a mutation that causes a defect in NCT includes mutations in TUBA4A, KIF5A, TDP 43, SOD1, kinesin, and Tau. A mutant PFN1 may comprise a mutation that corresponds to a C71G, M114T, G118V, A20T, T109M, Q139L, and/or E117G mutation in SEQ ID NO: 10. A mutation in C9ORF72 may comprise a repeat expansion. In some embodiments, a repeat expansion in C9ORF72 comprises GGGGCC repeats (e.g., 80 GGGGCC repeats; (G₄C₂)₈₀ (SEQ ID NO: 12)). In some embodiments, a repeat expansion in C9ORF72 comprises 5-100, 5-50, 25-100, 50-100, 50-80, 70-90, 80-200, or 100-500 GGGGCC (G₄C₂) repeats.

In some embodiments, a cell is isolated from a subject (e.g., a human subject suffering from a disease associated with a NCT defect). In some embodiments, a cell has been previously frozen and thawed (e.g., 1, 2, 3, 4, 5, or more freeze/thaw cycles). In some embodiments, a cell is maintained in liquid culture media. In some embodiments, a cell belongs to a population of cells (e.g., a population of motor neurons). In some embodiments, a cell belongs to a population of cells that has been passaged 1, 2, 3, 4, 5, or more times, using any known method.

Profilin-1 (NCBI Sequence: NP_005013.1; human)
(SEQ ID NO: 10)
MAGWNAYIDNLMADGTCQDAAIVGYKDSPSVWAAVPGKTFVNITPAEVGV
LVGKDRSSFYVNGLTLGGQKCSVIRDSLLQDGEFSMDLRTKSTGGAPTFN
VTVTKTDKTLVLLMGKEGVHGGLINKKCYEMASHLRRSQY

### Subjects

As used herein, a subject generally refers to any human subject. In some embodiments, a subject may be a human subject, a non-human primate subject, a pig subject, a rodent subject, or any suitable mammalian subject. A subject (e.g., a human subject) may be suffer from or otherwise have a disease associated with a nucleocytoplasmic transport (NCT) defect. In some embodiments, a subject has a neurodegenerative disease. In some embodiments, a subject having a disease associated with a NCT disease has Amyotrophic lateral sclerosis (ALS), Alzheimer's disease, Huntington's disease, or Frontotemporal dementia (FTD). In some embodiments, a subject has sporadic ALS or familial ALS. In some embodiments, a subject has a mutation in at least one gene or protein that causes or is correlated with a NCT defect. In some embodiments, a subject has a mutation in C9ORF72, PFN1, TUBA4A, KIF5A TDP 43, SOD1, kinesin, and/or Tau.

In some embodiments, a cell comprises at least one mutation that causes a defect in nucleocytoplasmic transport (NCT). In some embodiments, a mutation that causes a defect in NCT includes mutations in profilin-1 (PFN1). In some embodiments, a mutation that causes a defect in NCT includes mutations in the C9ORF72 gene. In some embodiments, a mutation that causes a defect in NCT includes mutations in TUBA4A, KIF5A, TDP 43, SOD1, kinesin, and Tau. A mutation PFN1 may comprise a mutation that corresponds to a C71G, M114T, G118V, A20T, T109M, Q139L, and/or E117G mutation in SEQ ID NO: 7. A mutation in C9ORF72 may comprise a repeat expansion. In some embodiments, a repeat expansion in C9ORF72 comprises GGGGCC repeats (e.g., 80 GGGGCC repeats; (G₄C₂)₈₀ (SEQ ID NO: 12)). In some embodiments, a repeat expansion in C9ORF72 comprises 5-100, 5-50, 25-100, 50-100, 50-80, 70-90, 80-200, or 100-500 GGGGCC (G₄C₂) repeats.

### Methods of Treatment

In some aspects, the disclosure provides methods for treating a subject having a disease associated with a nucleocytoplasmic transport (NCT) defect (e.g., Amyotrophic lateral sclerosis (ALS), Alzheimer's disease, Huntington's disease, or Frontotemporal dementia (FTD)). A subject can be a human, non-human primate, rat, mouse, cat, dog, or other mammal. In some embodiments, a method of treating a subject having a disease associated with a nucleocytoplasmic transport (NCT) defect comprises administering a molecular agent as disclosed herein that stabilizes the cytoskeleton (e.g., promotes polymerization of actin and/or tubulin; or inhibits actin and/or tubulin depolymerization) to the subject.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms, for example, symptoms associated with NCT defects. Thus, the terms denote that a beneficial result has been conferred on a subject having a disease associated with a nucleocytoplasmic transport (NCT) defect, or with the potential to develop such a disorder. Furthermore, treatment include the application or administration of a molecular agent to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease.

Therapeutic agents or therapeutic compositions may include a molecular agent as described (e.g., transgene, protein or small molecule) in a pharmaceutically acceptable form that prevents and/or reduces the symptoms of a particular disease (e.g., ALS, Alzheimer's disease, Huntington's disease, or FTD). For example a therapeutic composition may be a pharmaceutical composition that prevents and/or reduces the symptoms of a disease associated with a NCT defect. In some embodiments, the composition further comprises a pharmaceutically acceptable excipient. It is contemplated that the therapeutic composition of the present disclosure will be provided in any suitable form. The form of the therapeutic composition will depend on a number of factors, including the mode of administration as described herein. The therapeutic composition may contain diluents, adjuvants and excipients, among other ingredients as described herein.

Recombinant Adeno-Associated Viruses (rAAVs)

In some aspects, the disclosure provides isolated AAVs that are useful for delivering transgenes that encode proteins that stabilize the cytoskeleton (e.g., by promoting actin polymerization). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

In some aspects, the disclosure provides an rAAV having a capsid appropriate for targeting central nervous system (CNS) tissue or other tissue (e.g., a peripheral tissue). In some embodiments, the capsid has a serotype selected from the group consisting of AAV1, AAV2, AAV5, AAV6, AV6.2, AAV7, AAV8, AAV9 and AAVrh.10. In some embodiments, an rAAV described herein may comprise variants of AAV1, AAV2, AAV5, AAV6, AV6.2, AAV7, AAV8, AAV9, and AAVrh.10 serotype capsid proteins. In some embodiments, the capsid protein comprises an amino acid sequence that is at least 70%, at least 80%, at least 90%, at least 95%, or at least 99% identical to any one of the recited capsids.

Appropriate methods may be used for obtaining recombinant AAVs having a desired capsid protein. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types (e.g., AAV2, AAV3, AAV4, AAV5, or AAV6 ITR sequences).

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeats (ITRs) of one AAV serotype and an capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a gene associated with a neurodegenerative disease (e.g., a leukodystrophy). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The foregoing methods for packaging recombinant vectors in desired AAV capsids to produce the rAAVs of the disclosure are not meant to be limiting and other suitable methods will be apparent to the skilled artisan.

### Isolated Nucleic Acids

A “nucleic acid” sequence refers to a DNA or RNA sequence (e.g., comprising a transgene, e.g., a transgene encoding an enzyme that polymerizes actin, an actin-severing protein, an actin capping protein, or an actin bundling protein). In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the disclosure may comprise a vector or a plasmid. As used herein, a vector or plasmid may include any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, vector or plasmid includes cloning and expression vehicles, as well as viral vectors. In some embodiments, useful vectors are contemplated to be those vectors in which the nucleic acid segment to be transcribed is positioned under the transcriptional control of a promoter.

A vector or a plasmid may require an origin of replication, e.g., for replication of the vector or plasmid in a host. In some embodiments, a plasmid comprises an origin of replication that is maintained at a high copy number, e.g., from pUC18 or pUC19. In some embodiments, a plasmid comprises an origin of replication that is maintained at a medium copy number, e.g., derived from ColE1, e.g., from pETDuet. In some embodiments, a vector or a plasmid may further comprise a selection marker that ensures maintenance during growth on selective media. In some embodiments, a selection marker is a positive selection marker, e.g., a protein or gene that confers a competitive advantage to a bacterium that contains the selection marker. In some embodiments, a selection marker is a negative selection marker, e.g., a protein or gene that inhibits the growth and/or division of a bacterium that contains the selection marker. In some embodiments, a selection marker is an antibiotic resistance gene.

The nucleic acids of the disclosure may comprise an RNA, e.g., a messenger RNA (mRNA). In some embodiments, an mRNA may comprise a polyA tail at its 3′ end, e.g., a poly A-30 tail comprising 30 adenine bases. In some embodiments, a polyA tail comprises about 30, about 50, about 75, about 100, about 150, about 200, or about 300 adenine bases. In some embodiments, an mRNA may comprise a 5′ cap, e.g., a GAG cap or a 7-methylguanosine cap. In some embodiments, an mRNA may further comprise at least one untranslated region. In some embodiments, an mRNA may be single-stranded.

The nucleic acids of the disclosure may comprise a vector (e.g., a gene therapy vector). In some embodiments, a vector may be a viral vector (e.g., a lentiviral vector, an adeno-associated virus vector, etc.), a plasmid, a closed-ended DNA (e.g., ceDNA), etc. In some embodiments, a gene therapy vector is a viral vector. In some embodiments, an expression cassette encoding a transgene is flanked by one or more viral replication sequences, for example lentiviral long terminal repeats (LTRs) or adeno-associated virus (AAV) inverted terminal repeats (ITRS).

In some embodiments, a closed-ended DNA (ceDNA) vector comprises an expression cassette that comprises a cis-regulatory element, a promoter and a transgene. In some embodiments, a ceDNA comprises a promoter operably linked to a one transgene. In some embodiments, a ceDNA comprises an expression cassette comprising a transgene that is flanked by two self-complementary sequences (e.g., inverted terminal repeats) and is not associated with a capsid protein. In some embodiments, a ceDNA vector comprises two self-complementary sequences found in an AAV genome, wherein at least one comprises an operative Rep-binding element (RBE) and a terminal resolution site of AAV or a functional variant of the RBE, and one or more cis-regulatory elements operatively linked to a transgene. In some embodiments, a ceDNA vector is as described in WO2017/152149, published Sep. 8, 2019, or WO2019/051255, published Mar. 14, 2019; the contents of each of which are incorporated herein by reference.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., shRNA, miRNA) from a transcribed gene.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible, ubiquitous, and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and/or other vector elements may be performed, as appropriate, and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EFla promoter [Invitrogen]. In some embodiments, a promoter is an enhanced chicken 3-actin promoter. In some embodiments, a promoter is an astrocyte specific promoter. In some embodiments, a promoter is an oligodendrocyte specific promoter. In some embodiments, a promoter is an CNS-specific promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. In some embodiments, the promoter is an oligodendrocyte-specific promoter, for example the myelin basic protein (MBP) promoter (Chen et al., J. Neurosci, Res., 55(4); 504-13 (1999)).

### Pharmaceutical Compositions

In some aspects, the disclosure relates to pharmaceutical compositions comprising a molecular agent as described herein. In some embodiments, the composition comprises a molecular agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described herein. The active ingredients may be admixed or compounded with any conventional, pharmaceutically acceptable carrier or excipient. The compositions may be sterile.

Typically, pharmaceutical compositions are formulated for delivering an effective amount of an agent. In general, an “effective amount” of an active agent refers to an amount sufficient to elicit the desired biological response. An effective amount of an agent may vary depending on such factors as the desired biological endpoint, the pharmacokinetics of the compound, the disease being treated (e.g., ALS), the mode of administration, and the patient.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. It will be understood by those skilled in the art that any mode of administration, vehicle or carrier conventionally employed and which is inert with respect to the active agent may be utilized for preparing and administering the pharmaceutical compositions of the present disclosure.

An effective amount, also referred to as a therapeutically effective amount, of a molecular agent (e.g., a protein, transgene or small molecule as described herein) is an amount sufficient to ameliorate at least one adverse effect associated with a disease associated a NCT defect (e.g., ALS). In the case of viral vectors, an amount of active agent can be included in each dosage form to provide between about 10¹⁰, 10¹¹, 10¹², 10¹³, 10¹⁴, or 10¹⁵ genome copies per subject. One of ordinary skill in the art would be able to determine empirically an appropriate therapeutically effective amount.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above.

The compositions may conveniently be presented in unit dosage form. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. In some embodiments, liquid dose units are vials or ampoules. In some embodiments, solid dose units are tablets, capsules and suppositories.

### Modes of Administration

The molecular agents of the disclosure may be delivered to a subject in compositions according to any appropriate methods known in the art. For example, an rAAV or an mRNA comprising a transgene described herein, preferably suspended in a physiologically compatible carrier (e.g., in a composition), may be administered to a subject, e.g., a subject having a disease associated with a NCT defect.

Delivery of the compositions herein to a mammalian subject may be by, for example, intramuscular injection or by administration into the bloodstream of the mammalian subject. Administration into the bloodstream may be by injection into a vein, an artery, or any other vascular conduit. In some embodiments, the compositions are administered into the bloodstream by way of isolated limb perfusion, a technique well known in the surgical arts, the method essentially enabling the artisan to isolate a limb from the systemic circulation prior to administration of the compositions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, a composition as described in the disclosure are administered by intravenous injection. In some embodiments, compositions are administered by intracardiac injection. In some embodiments, compositions are administered by transcutaneous injection, intravascular injection, intramuscular injection, cardiopulmonary bypass, or a combination thereof.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

The compositions are administered in sufficient amounts to transfect the cells and to provide sufficient levels of gene transfer and expression without undue adverse effects. Conventional and pharmaceutically acceptable routes of administration include, but are not limited to, direct delivery to the selected organ (e.g., delivery to the CNS), oral, inhalation (including intranasal and intratracheal delivery), intraocular, intravenous, intramuscular, subcutaneous, intradermal, intratumoral, and other parental routes of administration. Routes of administration may be combined, if desired.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more molecular agents that stabilize the cytoskeleton (e.g., promotes polymerization of actin and/or tubulin; or inhibits actin and/or tubulin depolymerization) as described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1. Materials and Methods

Primary Motor Neuron Culture, Transfection, and Treatments

Primary motor neurons (MNs) were isolated from E12.5 mouse embryonic spinal cords dissociated in 0.1% trypsin (Worthington) at 37° C. for 12 minutes. MNs were purified using a 6% Optiprep (Sigma-Aldrich) density gradient and plated on glass coverslips coated with 0.5 g/L poly-ornithine (Sigma-Aldrich) and laminin (Thermo Fisher). Cells were grown at 37° C. and 5% CO₂ in Neurobasal medium (Thermo Fisher) supplemented with 0.25% Glutamax, 2% B27, 2% horse serum, and 10 ng/ml BDNF, GDNF, and CNTF. MNs at 2 days in vitro (DIV) were transfected using 1.75 μl NeuroMag reagent (OZ Biosciences)+0.5 μg DNA. Complete growth medium was replaced with serum free neurobasal medium 1 hour prior and after transfection. The paramagnetic nanobeads used for transfection can linger inside the cell and in the extracellular space for several days and can be detected as DAPI positive dots. The V5-PFN1 plasmids were described in Wu, C. H. et al. Mutations in the profilin 1 gene cause familial amyotrophic lateral sclerosis. Nature 488, 499-503 (2012). The (G₄C₂)₈₀ (SEQ ID NO:12). construct was described in Almeida, S. et al. Modeling key pathological features of frontotemporal dementia with C9ORF72 repeat expansion in iPSC-derived human neurons. Acta Neuropathol 126, 385-399 (2013). For the GFP-PFN1 constructs, WT or mutant PFN1 was PCR amplified from the V5 constructs and cloned in the pEGFP-C1 backbone using BamHI sites. The FH1-FH2 domains of murine formin mDia1 (amino acid residues 553-1192) were PCR amplified using specific primers and cloned into the pEGFP-C1 plasmid using XhoI and BamHI restriction sites. GFP or GFP-mDia1 were co-transfected in a 1:2 ratio with PFN1 or C9ORF72 plasmids. Mock transfection controls were performed by subjecting the cells to all the steps of transfection without adding any plasmid DNA. For actin depolymerization assays, 2 DIV MNs were treated with 0.1 mg/L Latrunculin A (Cayman) for 3 days. For nuclear export inhibition, MNs were treated with 50 nM KPT-276 (Selleck) or equal volume of DMSO (Sigma Aldrich) 1 day after transfection. For formin induction, MNs were treated with 0.1 μM IMM01 (EMD Millipore) for 24 hr before fixation at 6 DIV.

Lymphoblast Cells Culture

Immortalized lymphoblastoid cell lines obtained from 3 ALS patients carrying PFN1 mutations and 4 control cell lines were cultured in RPMI medium supplemented with 15% fetal bovine serum (FBS) for a maximum of 10 passages. Cells were seeded on a poly-ornithin coated coverslip at the density of 300 cell/mm². Thirty minutes after plating, cells were fixed in 4% paraformaldehyde and processed for immunofluorescence assays as described below.

Primary Cortical Neuron Culture, Transfection, and Treatments

Primary cortical neurons were isolated from E15 mouse embryos dissociated in 0.05% trypsin-EDTA (Thermo Scientific) at 37° C. for 12 minutes. 320,000 cells/ml were plated on poly-D-lysine (0.125 mg/ml, Sigma Aldrich) and lamin (5 μg/ml, Corning) coated 96-well glass plates and grown at 37° C. and 5% CO₂ in Neurobasal medium (Thermo Fisher) supplemented with 2% B27 and 1% Glutamax (Thermo Scientific). Four DIV neurons were transfected with 0.2 μl Lipofectamine 2000 (Thermo Scientific) and 100 ng DNA following manufacturer's recommendations. A ratio of 4:1 was used for the GFP and S-mCherry vectors. Thirty-six hours after transfection, Leptomycin B (10 mg/L) was added to the culture medium immediately before image acquisition. Cells were imaged using a Nikon TiE widefield microscope equipped with temperature- and CO₂-controlled environmental chamber. Movies were acquired with a 20× lens at a rate of 1 frame every 1 or 2 minutes for 1 hour. For rescue experiments, neurons were fixed immediately after acquisition and stained with V5 antibody to detect V5-PFN1 expression.

Fibroblast Culture, Treatment and Immunofluorescence

Fibroblasts were obtained from skin biopsies of 3 ALS patients carrying mutations in C9ORF72 gene and of 3 healthy donors, sex- and age-matched with ALS cases, after informed consent and approval by the IRCCS Istituto Auxologico Italiano ethics committee. C9ORF72 mutation was validated in primary fibroblasts by repeat-primed PCR and repeat expansion size determined by Southern blot analysis (range 600-1500 units). Fibroblasts were grown in RPMI 1640 medium (EuroClone) containing 2 g/L glucose and supplemented with 10% FBS (Sigma Aldrich), 2 mM L-glutamine, 2.5 μg/ml amphotericin B (Sigma Aldrich), 100 units/ml penicillin and 100 μg/ml streptomycin (Gibco). 10,000 cell/well were seeded on glass coverslip in 24-well plates and after 24 hours, 50% of medium was replaced with fresh medium and IMM01 (Sigma Aldrich) at the final concentration of 0.1 μM. After a 24-hour treatment, cells were fixed with 4% paraformaldehyde for 20 minutes, permeabilized with 0.3% Triton X-100/1×PBS for 5 minutes and processed for immunofluorescence as described below.

Immunofluorescence and Image Acquisition

Cells were fixed with 4% paraformaldehyde for 15 minutes. Fixed motor neurons were treated with hot 10 mM citrate buffer, pH 6 for 20 minutes before permeabilization with 0.2% Triton-X 100 for 5 minutes. Cells were blocked with 5% bovine serum albumin for 45 minutes and hybridized with the appropriate antibodies overnight at 4° C. Anti-mouse and anti-rabbit donkey secondary antibodies or phalloidin conjugated with either Alexa Fluor 647, Alexa Fluor 594, Alexa Fluor 555, Alexa Fluor 546, or Alexa Fluor 488 (Jackson Immunoresearch and Thermo Fisher) were hybridized for 1 hour at room temperature. Coverslips were mounted onto a glass slide using Prolong Gold mounting medium (Thermo Fisher) or FluorSave mounting medium (Calbiochem) and imaged using an epifluorescence microscope (Nikon Ti E) equipped with a cooled CMOS camera (Andor Zyla) or a Digital sight DS-U3 camera. Images were acquired as Z-stacks (0.2 μm step size) using a 60× lens unless otherwise specified. For propidium iodide (PI) staining, cells were incubated with 20 μg/ml PI (Thermo Fisher) for 30 minutes at 37° C. before fixation. As a positive control, cells were heat shocked at 65° C. for 30 minutes before incubation with PI.

Transmission Electron Microscopy

Six-well plates of cultured cells were fixed overnight at 4° C. by adding 1 ml of 2.5% glutaraldehyde in 0.1 M sodium cacodylate-HCl buffer (pH 7.2). Fixed samples were washed three times in 0.5 M sodium cacodylate-HCl buffer (pH 7.0) and post-fixed for 1 hour in 1% osmium tetroxide (w/v) in the same buffer at room temperature. Following post-fixation, the culture dish with adherent cells were enblock stained (20 min) with 1% aqueous uranyl-acetate (w/v). The fixed cell culture dishes were washed again in the same buffer and dehydrated through a graded series of 0% ethanol to 100% and transferred through two changes of 50/50 (v/v) SPIpon resin (Structure Probe, Inc.)/100% ethanol and left overnight to infiltrate. The following morning, the cell culture plates were transferred through three changes of fresh SPIpon resin to finish the infiltration and embedding and finally, the dishes were filled with freshly prepared SPIpon resin and polymerized for 48 hours at 70° C. Once fully polymerized, the six well-plate was cut apart and each well was plunged into liquid nitrogen to separate the SPIpon epoxy block with the embedded cells from the culture dish. The round epoxy disks with the embedded cells were then examined under an upright light microscope and areas of cells were cut from the disks and glued onto blank microtome studs and trimmed for ultramicrotomy. Ultrathin sections (70 nm) were cut on a Reichart-Jung ultramicrotome using a diamond knife. The sections were collected and mounted on copper support grids and contrasted with lead citrate and uranyl acetate and examined on a FEI Tecnai G2 Spirit transmission electron microscope at 100 Kv accelerating voltage and images were recorded at various magnifications using a Gatan 2 K digital camera system.

Fluorescence In Situ Hybridization

Motor neurons were fixed in 4% RNase-free paraformaldehyde for 15 minutes and stored in 70% ethanol at 4° C. overnight. Cells were sequentially incubated for 5 minutes in 1×PBS and wash buffer (2×SSC, 10% formamide), and hybridized in hybridization buffer (10 mg/ml dextran sulfate, 4 mg/ml BSA, 40 μM ribonucleoside vanadyl complexes, 2×SSC, 1% PBS) at 37° C. overnight with 12.5 μM probes and 0.2 mg/ml each salmon sperm DNA and E. Coli tRNA. Neurofilament L mRNA specific probes were design using Biosearch Technology online tool and conjugated with the Quasar® 570 fluorophore. Coverslips were then washed twice for 30 minutes at 37° C. in wash buffer before mounting them as described above. FISH for C9ORF72 sense RNA hexanucleotide repeat was performed using the LNA probe 5′TYE563/CCCCGGCCCCGGCCCC (SEQ ID NO: 13) (Exiqon) as described in Chew, J. et al. Neurodegeneration. C9ORF72 repeat expansions in mice cause TDP-43 pathology, neuronal loss, and behavioral deficits. Science 348, 1151-1154 (2015).

Axon Length and Outgrowth Analysis

Motor neurons were co-transfected as outlined above with green fluorescent protein (GFP) and V5-tagged PFN1 constructs in a 1:2 ratio. KPT-276 (50 nM) or equal volumes of DMSO were added to the culture medium 1 day after transfection and maintained throughout the experiment for a total of 3 days. Cells were fixed and stained to detect V5-PFN1 expression 4 days after transfection. Cells were imaged as individual focal planes using a 10× lens. GFP was used to identify transfected cells and highlight the whole cell structure. For live imaging of axon outgrowth, KPT-276 (50 nM) or equal volumes of DMSO were added to the culture medium 1 day after transfection and maintained throughout the experiment for a total of ˜18-24 hours. Cells were imaged at 3 DIV using a Nikon TiE widefield microscope equipped with temperature- and CO₂-controlled environmental chamber. Movies were acquired with a 20× lens at a rate of 1 frame every 10 minutes for 1 hour.

Immunoprecipitations, Solubility Assays, and Western Blots

HEK293 cells were grown in DMEM+10% FBS were transfected with V5-PFN1 constructs and lysed with lysis buffer (20 mM Tris, 150 mM NaCl, 1% Triton X-100, protease inhibitor Complete EDTA-free, Roche) 24 or 48 hours after transfection. For immunoprecipitations, the detergent-soluble lysates were added to 30 μL of Protein A-Agarose beads (Roche) and 0.3 μg of anti-V5 antibody (Novus) and rocked overnight at 4° C. Immunoprecipitated complexes were eluted in Laemmli buffer (60 mM Tris-Cl pH 6.8, 2% SDS, 10% glycerol, 5% beta-mercaptoethanol, 0.01% bromophenol blue) and then subjected to western blot analysis. For solubility assays, lysates were sonicated on ice and then centrifuged at 16,000×g for 10 min at 4° C. The supernatant was collected as the soluble fraction. The pellet was washed three times with lysis buffer, resuspended in 8 M urea, sonicated, and then centrifuged at 16,000×g for 10 min at 4° C. The supernatant was collected as the insoluble fraction.

Samples were resolved by SDS-PAGE on Mini Protean TGX 4-20% gradient polyacrylamide gels (Bio-Rad) and transferred onto nitrocellulose membranes (Bio-Rad). Membranes were blocked with Odyssey Blocking Buffer (LI-COR) and probed with primary antibodies overnight. Secondary antibodies conjugated with IRDye® infrared fluorophores (LI-COR) were incubated for 1 hour at room temperature. Blots were visualized using the Odyssey Infrared Imaging System (LI-COR).

POLDIP3 Splicing Assay

Whole RNA was extracted from 5×10⁶ lymphoblast cells using TriZol reagent (Thermo Fisher) according to manufacturer's instructions. RNA (2 μg) was retrotranscribed using the High-Capacity cDNA Reverse Transcription Kit (Thermo Fisher). RT-PCR was performed using specific primers amplifying exon2-exon4 of human POLDIP3 mRNA. DNA gels were stained with SYBR Safe dye (Thermo Fisher) and imaged using the ChemiDoc XR+ imager (BioRad).

Image Processing and Quantification

Immunofluorescence images were deconvolved using an adaptative blind deconvolution algorithm (Autoquant X3, Media Cybernetics) before analysis. To measure fluorescence intensities, the signals were thresholded and the resulting integrated densities were normalized on the area of the selected region (e.g. cell body, nucleus). Thresholds were kept consistent for all images within experiments. For all experiments, values were normalized to WT PFN1 averages so that WT PFN1 always=1±SEM. For NCT dynamics, the fluorescence intensity of S-mCherry was measured in the nucleus using ImageJ and normalized to the background for every time point. All values were subsequently normalized to TO. For the analysis of axonal fluorescence intensities, a 100 μm long region of the proximal axon was measured in all conditions. Axon lengths were measured using the ImageJ plugin NeuronJ 60. The axon was defined as the longest neurite. The rate of axon outgrowth was measured using the ImageJ plugin MTrackJ by tracking the movement of the growth cone in all fields. A blinded analysis was performed to assess RanGAP1, RanBP2, POM121, F-Nups, and Lamin A/C localization. For nucleoporin analysis, abnormal staining was considered if the signal was not uniformly distributed around the nucleus with the presence of empty bare segments. For Lamin A/C, abnormal staining was defined as the absence of a think and uniform layer around the nucleus. DAPI was used as a reference for nuclear boundary. For all experiments, raw values were normalized to the mean of the control condition.

Nuclear Dextran Assay

Nuclei were isolated from lymphoblasts and then incubated with fluorescently labeled-Dextrans as described with minor modifications. Briefly, 10⁶ cells were pelleted by centrifugation for 5 minutes at 4° C. at 2600 RPM and resuspended in 1 mL of sucrose buffer (0.32 M sucrose, 3 mM CaCl₂), 2 mM Magnesium Acetate, 0.1 mM EDTA, 10 mM Tris HCl, 1 mM Dithiothreitol, and protease inhibitors (Roche, cat #11873580001)) with 1% NP-40. After 20 minute incubation on ice, the cells were washed in sucrose buffer and centrifuged for 5 minutes at 2600 RPM at 4° C. The pellet was washed in 1 mL of TR buffer (20 mM HEPES, 110 mM KOAc, 2 mM Mg(OAc)₂, 5 mM NaOAc, 0.5 mM EGTA, 250 mM sucrose, and protease inhibitors) and the isolated nuclei were then incubated for 30 minutes in 100 μL of TRB containing 0.6 mg/mL of 70 KDa RITC-Dextran (Millipore Sigma, R9379), 0.6 mg/mL of 500 KDa FITC-Dextran (Millipore Sigma, 46947), and Dapi and imaged using confocal microscopy. Images were analyzed using imageJ. RITC-dextran intensity in the nucleus was defined as the ratio of the nuclear mean intensity to the background intensity. Nuclei in which the RITC-dextran intensity was greater than the mean RITC-dextran intensity of all control nuclei plus 2 standard deviations were classified as leaky nuclei.

Statistical Analysis

Statistical analyses were performed using Prism 8 software package (GraphPad). Normality of the samples was assessed using the D'Agostino & Pearson Omnibus test. According to normality, parametric or non-parametric tests were used to assess significance, defined as p<0.05.

TABLE 2
Primers

| | | |
|---|---|---|
| POLDIP3_fwd | 5′-gcttaatgccagaccggg | SEQ ID |
| | agttgga-3′ | NO: 13 |
| POLDIP3_rev | 5′-tcatcttcatccaggtca | SEQ ID |
| | tataaatt-3′ | NO: 14 |
| GFP-PFNl_fwd | 5′-ggtggctctggaggcgga | SEQ ID |
| | tccgccgggtggaacgcctac | NO: 15 |
| | atcg-3′ | |
| GFP-PFNl_rev | 5′-ttatctagatccggtgga | SEQ ID |
| | tcctcagtactgggaacgccg | NO: 16 |
| | aagg-3′ | |
| GFP-mDial^{FH1-FH2}_fwd | 5′-gagactcgagccatggct | SEQ ID |
| | tctctctctgctg-3′ | NO: 17 |
| GFP-mDial^{FH1-FH2}_rev | 5′-gagaggatccttagcttg | SEQ ID |
| | cacggccaac-3′ | NO: 18 |

### Example 2. Mutations in PFN1 Impair Nucleocytoplasmic Transport

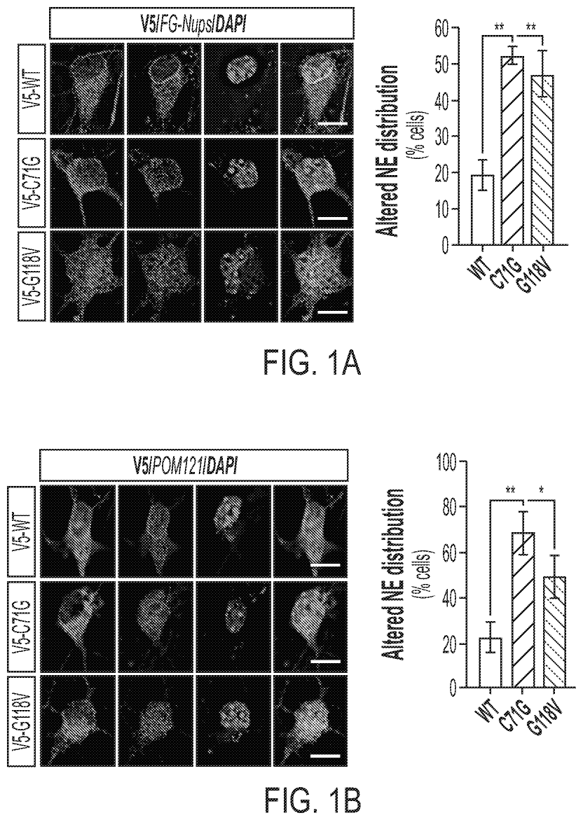

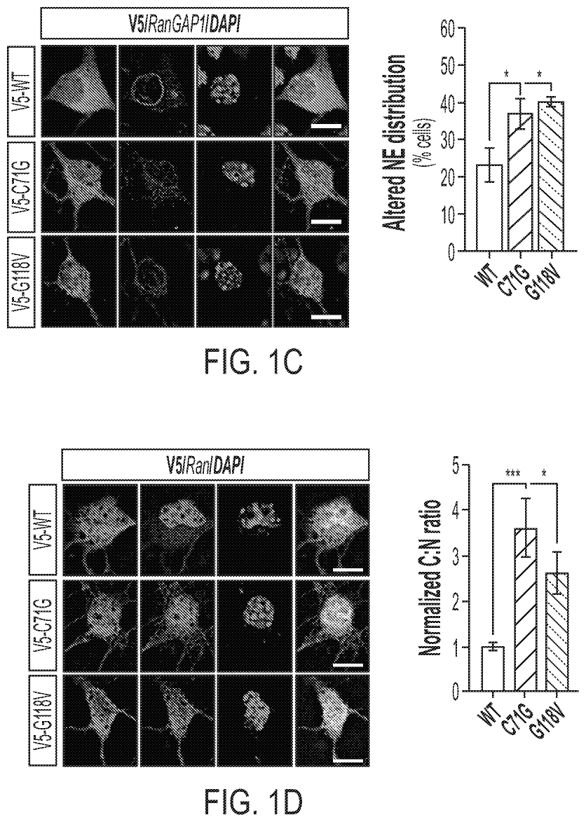

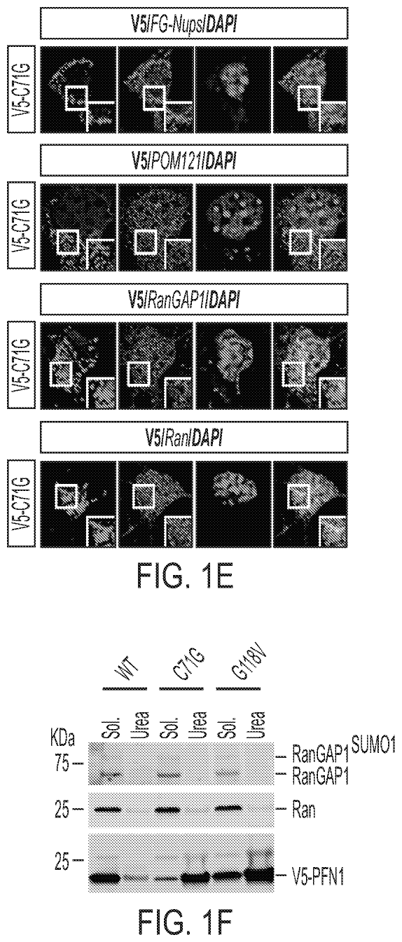

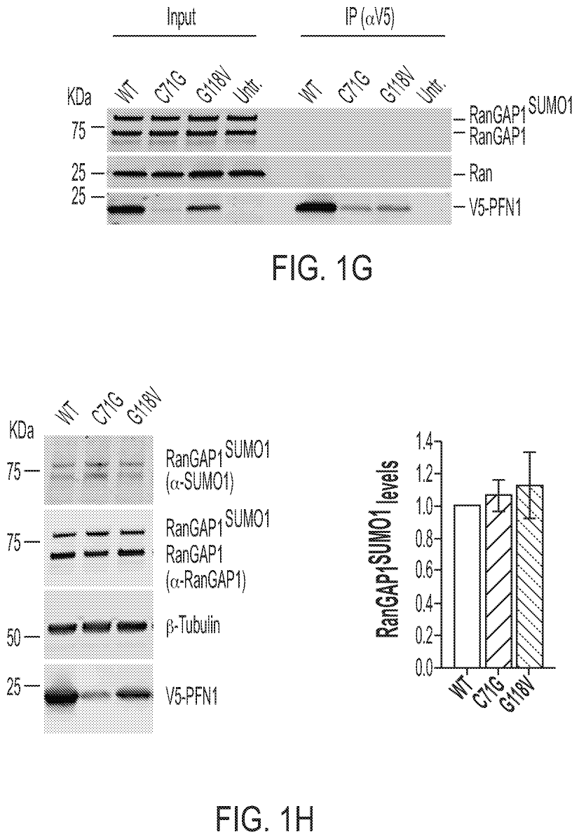

To investigate whether mutant PFN1 toxicity is associated with nucleocytoplasmic transport (NCT) defects, its effects on the distribution of essential factors controlling this process were examined. Wild type (WT) or mutant (C71G or G118V) V5-tagged PFN1 were transfected in primary motor neurons (MNs) for 4 days. Similar cellular distribution and expression was observed for all constructs. No effect on cell survival was evident at this time point due to the expression of mutant PFN1. To visualize the localization and composition of the nuclear pore complex (NPC) along the nuclear envelope (NE), MNs expressing WT or mutant PFN1 were stained with antibodies recognizing (1) nucleoporins of the FG-Nup family (i.e. Nup62, Nup153, Nup214, and Nup358; mAb414 24), (2) Nup358/RanBP2, and (3) the transmembrane Nup POM121. In WT PFN1 cells, all nucleoporins examined displayed a strong, punctate staining around the nucleus, as identified by DAPI staining, similar to mock transfected controls. In contrast, a significantly higher percentage of mutant PFN1 MNs showed reduced or absent staining at the NE (FIGS. 1A-B). RanGAP1 localized along the NE in WT PFN1 cells, while its staining pattern was partially or completely disrupted in mutant PFN1 MNs (FIG. 1C). The presence of mutant PFN1 led the transport factor Ran to be abnormally redistributed to the cytoplasm, in contrast to its mostly nuclear localization in WT PFN1 cells (FIG. 1D). This effect was more pronounced in cells containing visible inclusions, although MNs with no obvious aggregates still had Ran cytoplasm:nucleus (C:N) ratios significantly higher than WT PFN1 values. No co-aggregation of any of the tested proteins with C71G PFN1-positive inclusions was observed by immunofluorescence, detected by V5-staining (FIG. 1E), solubility assay (FIG. 1F), or co-immunoprecipitation (FIG. 1G). In addition, no changes in RanGAP1 SUMOylation, which is necessary for its association with the NPC 28, were detected (FIG. 1H). Similarly, no difference in the overall levels of the tested nucleoporins was observed in all conditions, while a slight reduction in Ran levels was present in C71G PFN1 MNs (data not shown). There were no observed changes to the localization of karyopherins Exportin 1 (XPO1) and Importin-β, with the exception of a small reduction in XPO1 levels. In all, these data demonstrate that, in the presence of mutant PFN1, NPCs are either reduced in number or structurally compromised because of the lack of essential nucleoporins, and additional key players in NCT are abnormally distributed.

### Example 3. Mutant PFN1 Alters the Structure of the Nuclear Membrane

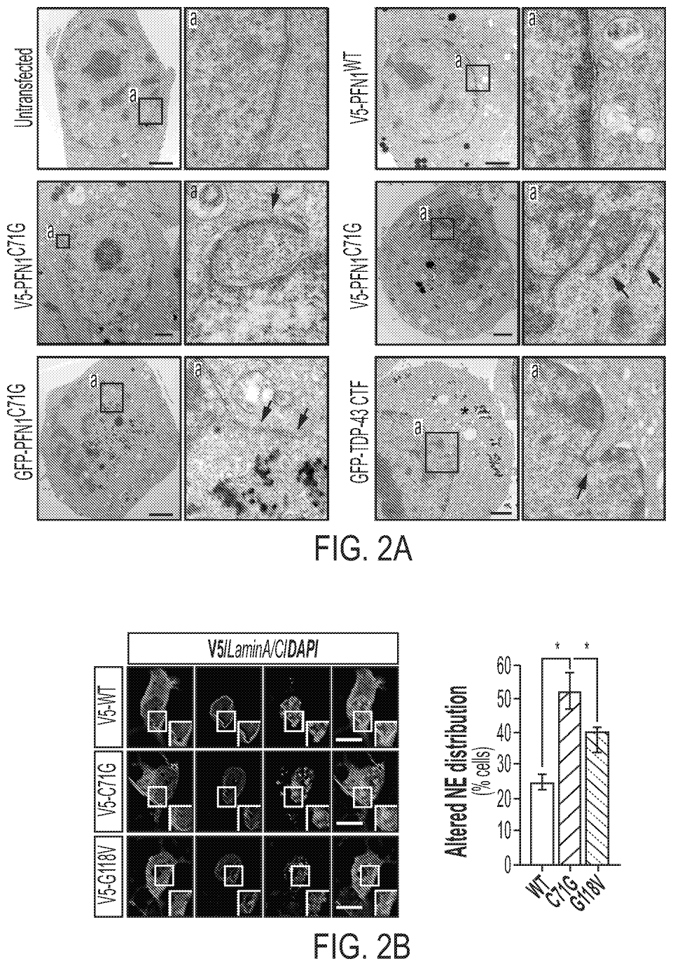

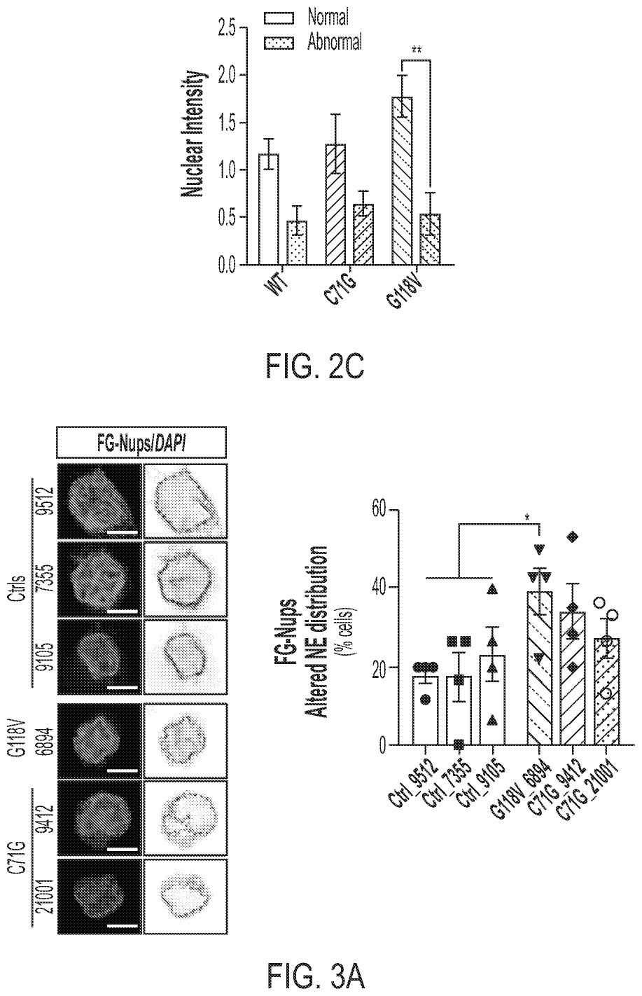

The effect of mutant PFN1 on the nuclear structure was further evaluated using transmitted electron microscopy. Similarly to what was observed in cells expressing TDP-43 C-terminal fragment 1, it was found that the expression of either V5-tagged or GFP-tagged C71G PFN1 in Neuro2a cells led to severe defects in the structure of the nucleus, with the presence of frequent folds, invaginations, and protrusions that were never observed in untransfected or WT PFN1-transfected N2a cells (FIG. 2A). This observation was confirmed by immunofluorescence analysis of Lamin A/C, one of several proteins constituting the nuclear lamina such as Lamin B1 and B2, emerin, Lamin B receptor, Nurim, MAN1, LAP1A-C, and LAP229. This analysis showed non-uniform and irregular staining in cells expressing mutant PFN1 (FIG. 2B). It was also found that cells with abnormal Lamin A/C staining were characterized by reduced nuclear levels of the protein (FIG. 2C) but no increase in cytoplasmic levels (data not shown), suggesting that Lamin A/C may be targeted for degradation as a consequence of its altered localization.

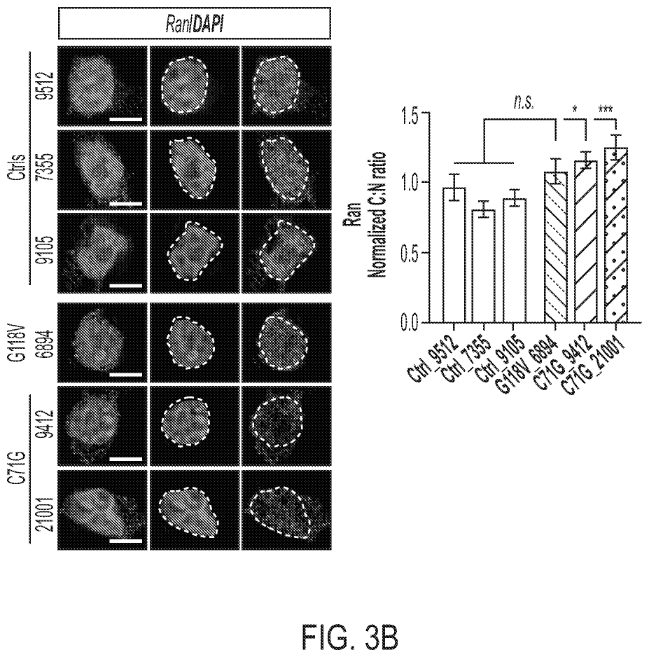

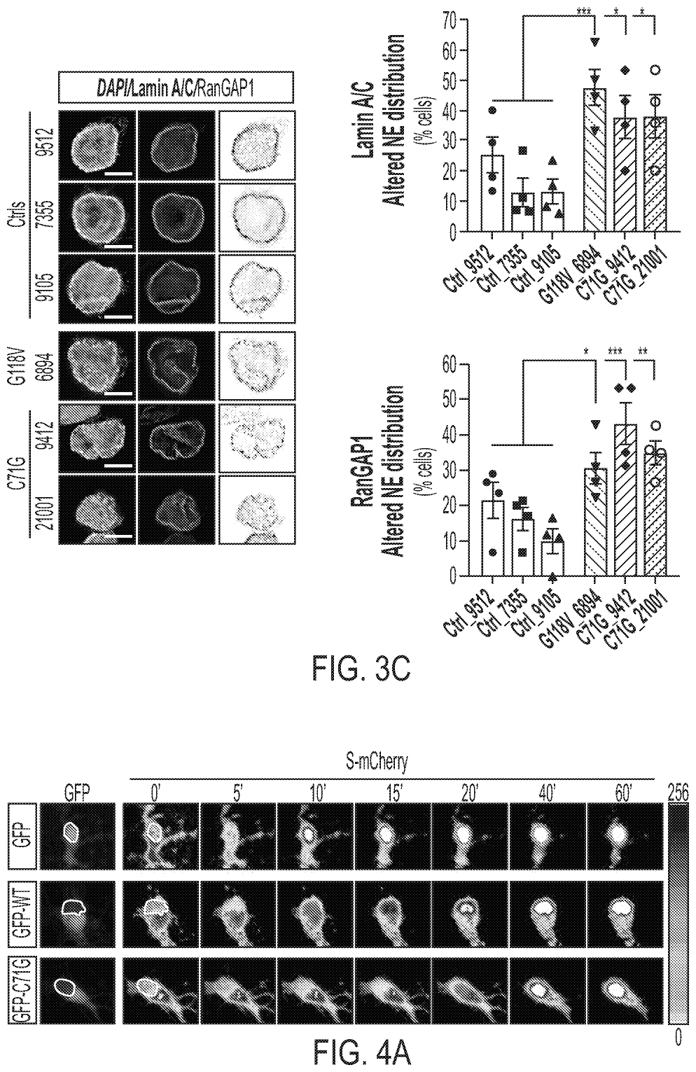

To verify that the effect of mutant PFN1 on the nuclear pore occurs also in human ALS patient cells immunofluorescence assays in immortalized lymphoblast cells derived from 3 controls and 3 patients carrying either the C71G or G118V PFN1 mutation were performed. Analyses of the localization and staining pattern of FG-Nups, RanGAP1, Ran, and Lamin A/C were performed on all lymphoblast lines. It was found that the percentage of cells with abnormal staining of Lamin A/C and RanGAP1 was significantly increased in all three lines endogenously expressing mutant PFN1 (FIG. 3), while FG-Nups—recognized by the mAb414 antibody (AbCam)—showed a significant change only in the G118V PFN1 cell line. Further, it was found that the nucleocytoplasmic localization of Ran was significantly altered in the C71G PFN1 cell lines. No changes in the overall levels of all proteins tested, including endogenous PFN1, were detected. To test whether the changes in Ran localization were caused by alteration to the integrity of the nuclear membrane, the ability of a large inert molecule (70 KDa-Dextran) to bypass the NPC and accumulate in the nucleus was determined. No increase in the presence of leaky nuclei was observed suggesting that disruption to nuclear membrane integrity is not an early event caused by mutant PFN1. Together, these data show that endogenous levels of mutant PFN1 directly affect the nuclear pore structure/stability in ALS patient cells, possibly leading to neuronal degeneration.

### Example 4. Nuclear Import is Greatly Reduced by Mutant PFN1

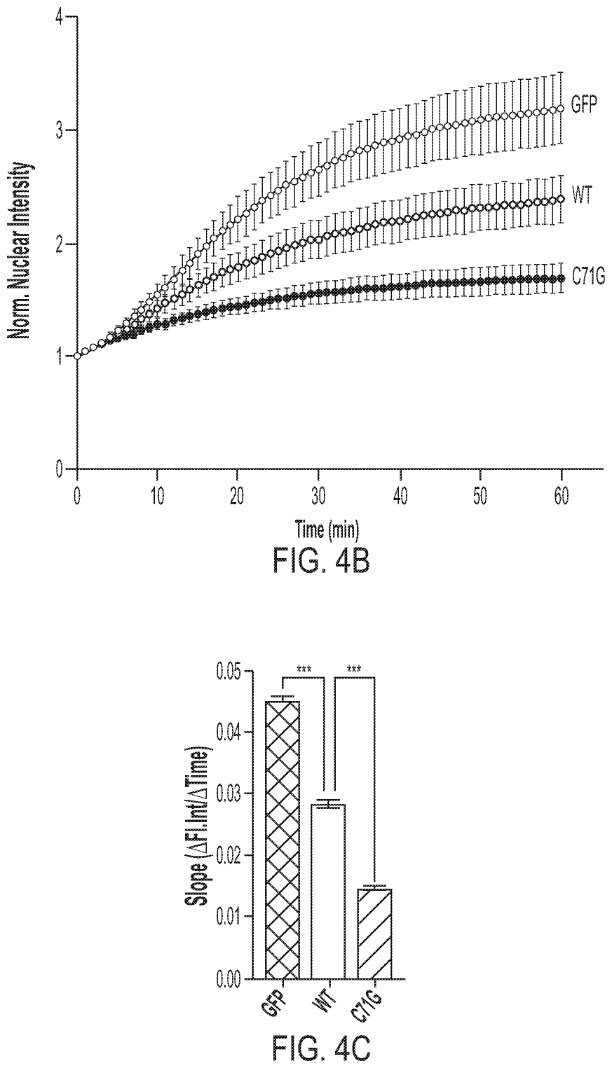

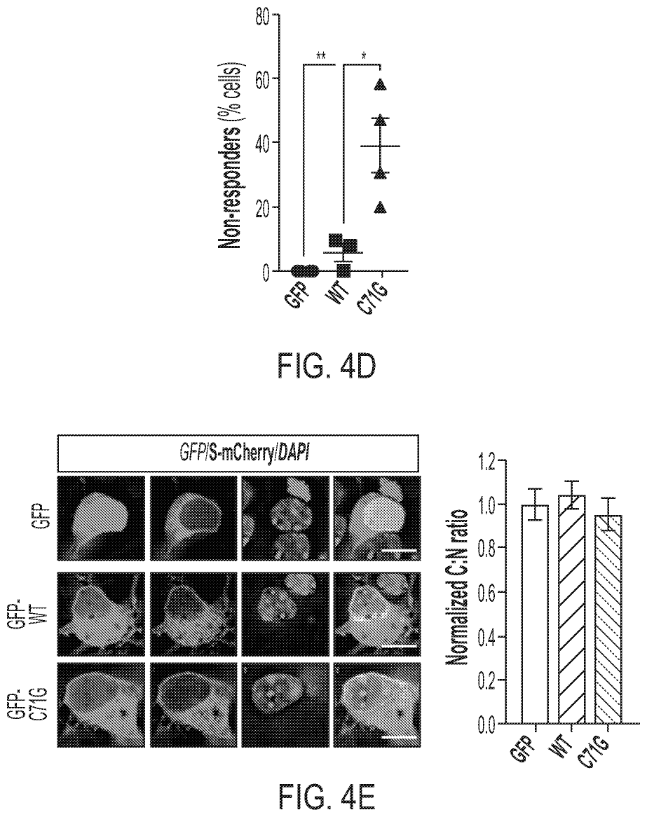

To further explore the functional consequences of the structural defects caused by mutant PFN1 on the NCT, the rate of nuclear import by live cell imaging using a NLS-NES-mCherry (Shuttling (S)-mCherry) reporter was measured. Cortical neurons were co-transfected with GFP or GFP-tagged PFN1 and S-mCherry. S-mCherry localized mainly to the cytoplasm in all conditions due to the stronger effect of the nuclear export signal (NES) compared to the nuclear localization signal (NLS) (FIG. 4E). Thirty-six hours after transfection, cells were treated with Leptomycin B—a selective inhibitor of Exportin 1—to inhibit nuclear export, leading to a measurable time-dependent accumulation of the reporter in the nucleus. We found that the expression of mutant PFN1 led to a significant reduction in import rates compared to both GFP- and WT PFN1-transfected cells (FIGS. 4A-4C), and an increase in the percentage of non-responder cells (39% in C71G vs 6% WT) (FIG. 4D). Expression of WT PFN1 also reduced import rates compared to the GFP control, but to a lesser degree compared to mutant PFN1. In all, these data show that mutant PFN1's ability to affect nuclear stability by altering the composition and/or the number of functional NPCs, leading to severe nuclear import defects.

### Example 5. mRNA Post-Transcriptional Regulation is Impaired in Mutant PFN1 Cells

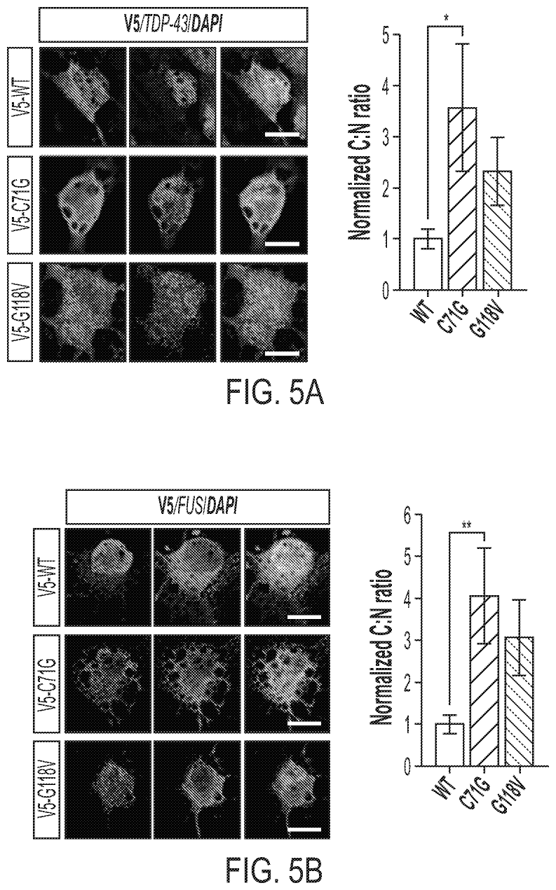

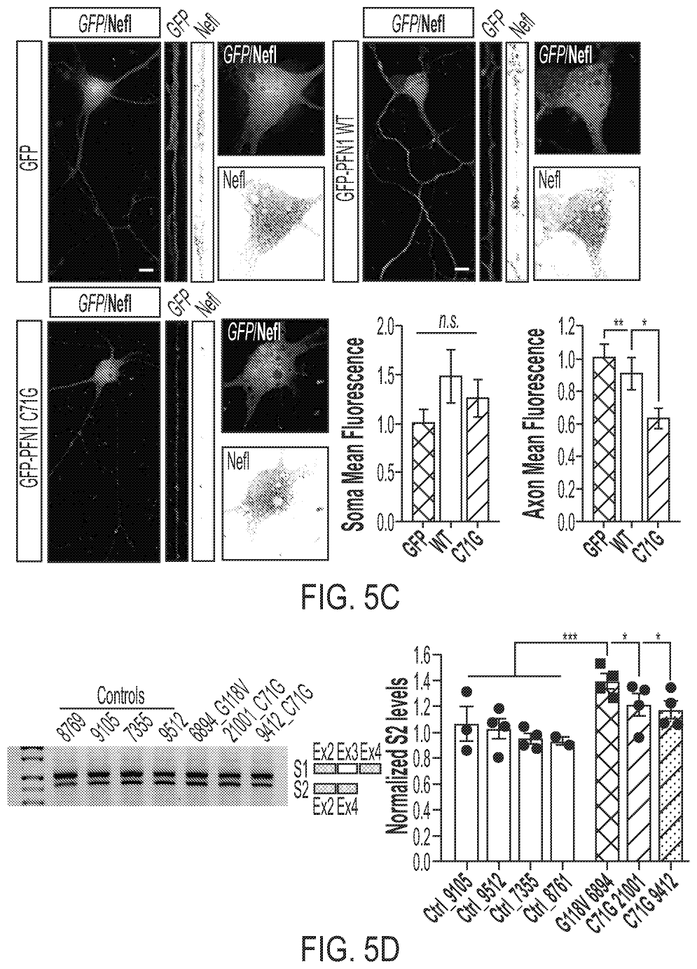

One of the main classes of proteins that shuttle between the nucleus and the cytoplasm are RBPs which control the post-transcriptional fate of mRNAs. The impact of mutant PFN1-dependent disturbance to NCT on the distribution of RBPs was determined by quantifying the nuclear and cytoplasmic levels of the mostly nuclear proteins TDP-43 and FUS, and of the mostly cytoplasmic proteins SMN and FMRP. The majority of TDP-43 and FUS was nuclear in WT-expressing MNs, while mutant PFN1 expression led to a shift in the C:N ratio of both proteins (FIGS. 5A-5B). No effect of WT PFN1 expression was observed on the localization of the proteins compared to untransfected controls (data not shown). On the contrary, there was no quantifiable changes in the distribution of the mostly cytoplasmic FMRP and SMN proteins, although fewer SMN-positive nuclear gems were observed. It was also observed that a significant reduction in TDP-43 localization to the proximal motor axon and an increase in TDP-43 aggregation was possibly mediated by TDP-43's illegitimate interaction with mutant PFN1. Together, these data show that mutant PFN1 perturbs the distribution of ALS-relevant nuclear RBPs by destabilizing the NPC and impairing nuclear import. The regulatory activity of TDP-43 on the axonal localization of the neurofilament L (Nef1) mRNA 31 and on the splicing of POLDIP3 pre-mRNA was tested. Quantitative fluorescence in situ hybridization was performed in MNs expressing either GFP or GFP-tagged WT or C71G PFN1 (FIG. 5C). While no difference in the somatic levels of the Nef1 mRNA was detected, its axonal levels were severely reduced in C71G-expressing MNs. Immortalized lymphoblast cell lines derived from 4 controls and 3 patients carrying PFN1 mutations were used to evaluate the abundance of two alternatively spliced POLDIP3 variants—S1 and S2—by RT-PCR (FIG. 5D). In all ALS lines, the relative levels of isoform S2 were significantly increased over control cells. Together these data show a loss of function for TDP-43 in mutant PFN1 cells, possibly due to its nucleocytoplasmic redistribution.

TABLE 3
Sequence information for the
Nefl mRNA FISH probes

| | | SEQ ID |
|---|---|---|
| Probe # | Sequence | NO: |
| msNefl_1 | gggggacctagagagaagaa | 20 |
| msNefl_2 | cgtagccgaacgaactcatg | 21 |
| msNefl_3 | cgcttgtaggaggtcgaaaa | 22 |
| msNefl_4 | agtagctggagtacgcggag | 23 |
| msNefl_5 | acggacagcgaggaggagac | 24 |
| msNefl_6 | atcaaagagccagagctgga | 25 |
| msNefl_7 | ctcagatcgagattctccag | 26 |
| 3msNefl_8 | ggatagacttgaggtcgttg | 27 |
| msNefl_9 | atgaagctggcgaagcgatc | 28 |
| msNefl_10 | gaaggctcagagtgtttctg | 29 |
| msNefl_11 | ttctcgttagtggcgtcttc | 30 |
| msNefl_12 | tcagcacttcttcctcatag | 31 |
| msNefl_13 | aaagctatctcgtccatcag | 32 |
| msNefl_14 | tctgagcatactggatctga | 33 |
| msNefl_15 | ttggaggacacgtccatctc | 34 |
| msNefl_16 | ttgaaccactcttcggcgtt | 35 |
| msNefl_17 | tctcggttagcacggtgaag | 36 |
| msNefl_18 | ttcgatctccagggtcttag | 37 |
| msNefl_19 | ctaatgtctgcattctgctt | 38 |
| msNefl_20 | tctccagtttgttgattgtg | 39 |
| msNefl_21 | catcttgacattgaggaggt | 40 |
| msNefl_22 | ctgcaatctcgatgtccaag | 41 |
| msNefl_23 | ccttccaagagttttctgta | 42 |
| msNefl_24 | tgaaactgagcctggtctct | 43 |
| msNefl_25 | aagacctgcgagctctgaga | 44 |
| msNefl_26 | aagccactgtaagcagaacg | 45 |
| msNefl_27 | gagcgagcagacatcaagta | 46 |
| msNefl_28 | cagctttcgtagcctcaatg | 47 |
| msNefl_29 | ttgggaatagggctcaatct | 48 |
| msNefl_30 | attggggagaacttttcctg | 49 |
| msNefl_31 | tgtataggatctggaactca | 50 |
| msNefl_32 | cctaagtcatctcagaatta | 51 |
| msNefl_33 | tagcacaacattgaaagtcc | 52 |
| msNefl_34 | gatactctgcgtaaggagga | 53 |
| msNefl_35 | aaagccactctgcaagcaaa | 54 |
| msNefl_36 | ataagcatggaccatgcaca | 55 |

### Example 6. Inhibition of Nuclear Export Improves ALS Relevant Disease Phenotypes

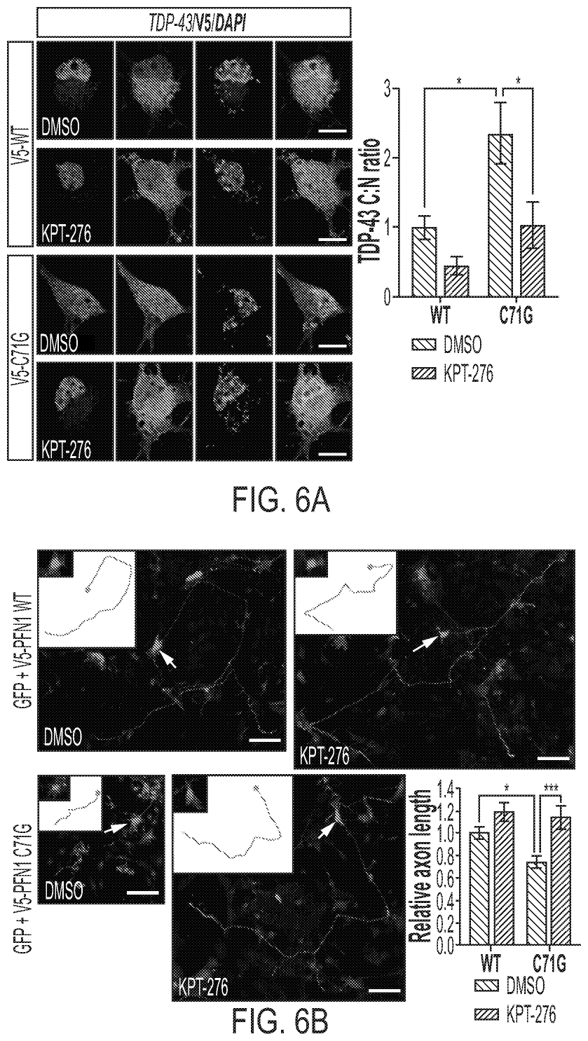

To assess the causality between NCT disturbance, RBP mislocalization, and MN pathology, the potential of nuclear export inhibitor KPT-276, a selective inhibitor of XPO1 was investigated for its ability to rescue PFN 1-dependent defects. First, WT PFN1 or C71G PFN1 expressing MNs were treated with 50 nM KPT-276 or DMSO 6 hours prior to fixation, and the C:N ratio of TDP-43 was determined. KPT-276 treatment was able to fully rescue TDP-43 cytoplasmic mislocalization (FIG. 6A), demonstrating successful inhibition of nuclear export. Next, the potential of KPT-276 treatment to rescue axonal outgrowth defects previously described in mutant PFN1 MNs was determined. MNs expressing mutant PFN1 and treated with vehicle alone had significantly shorter axons compared to WT PFN1 cells, while KPT-276 treatment fully rescued the defect (FIG. 6B). Similar rescue was observed by measuring the rate of axon growth by live cell imaging over the course of 1 hour (FIG. 6C). Together, these data support a direct link between NCT, mRNA regulation, and PFN1-dependent ALS cellular defects.

### Example 7. Modulation of Actin Polymerization Modifies NCT in Mutant PFN1 MNs

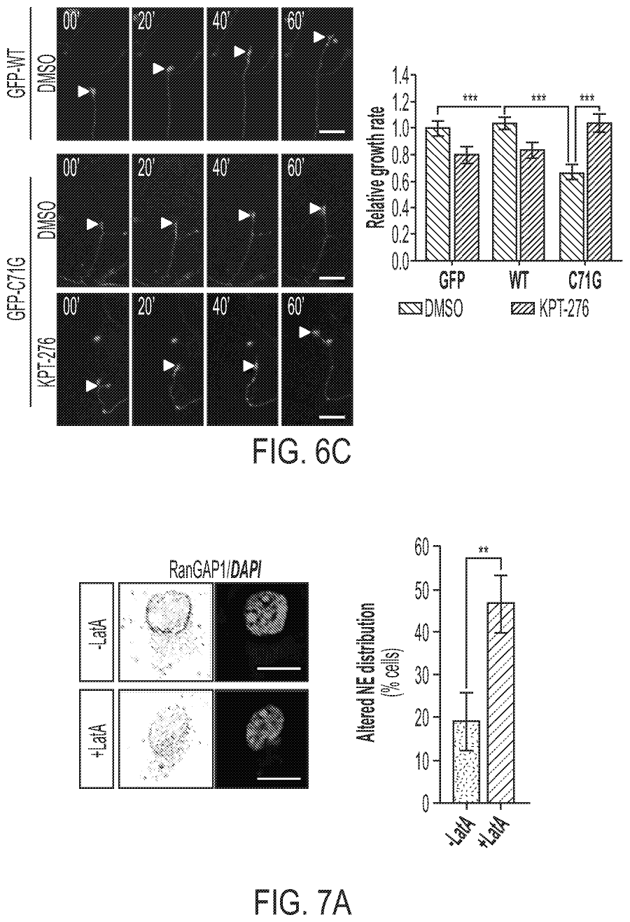

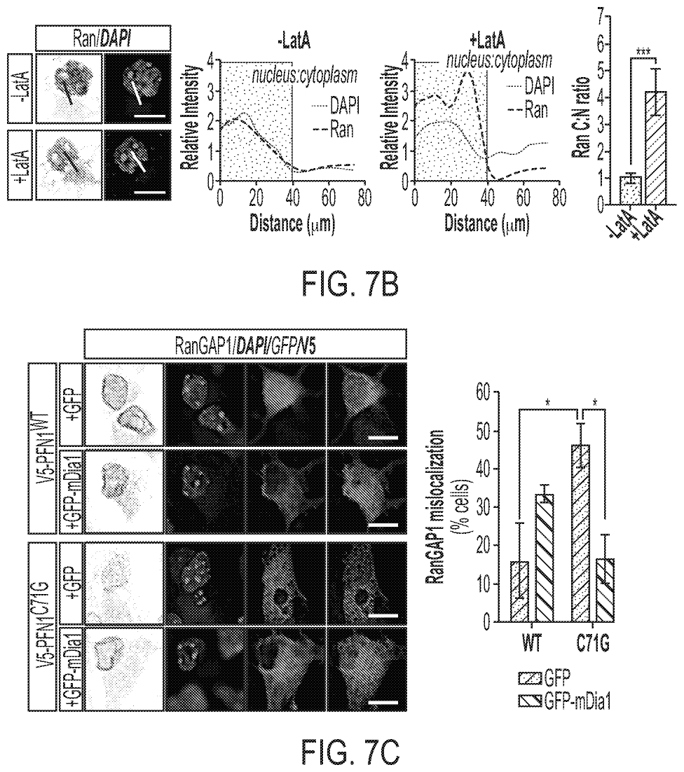

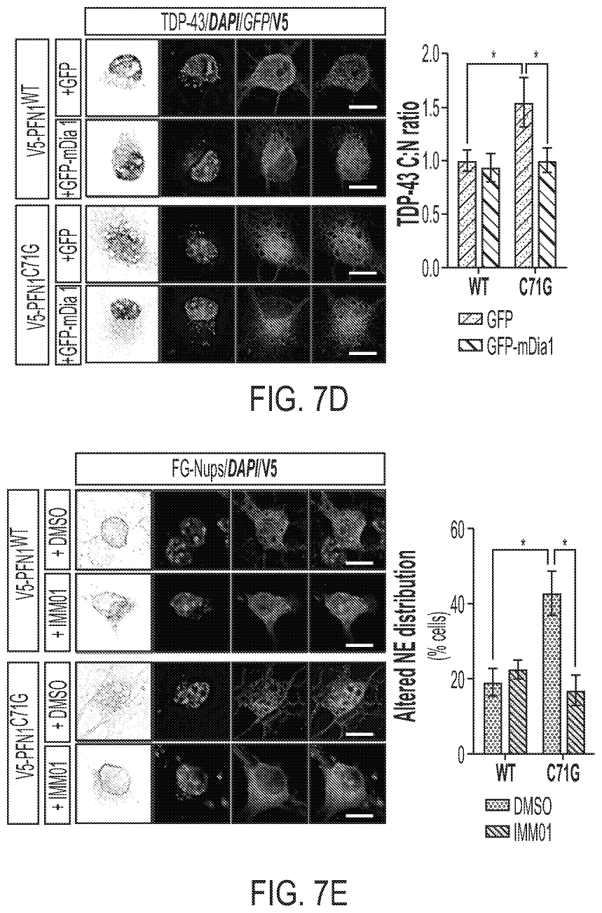

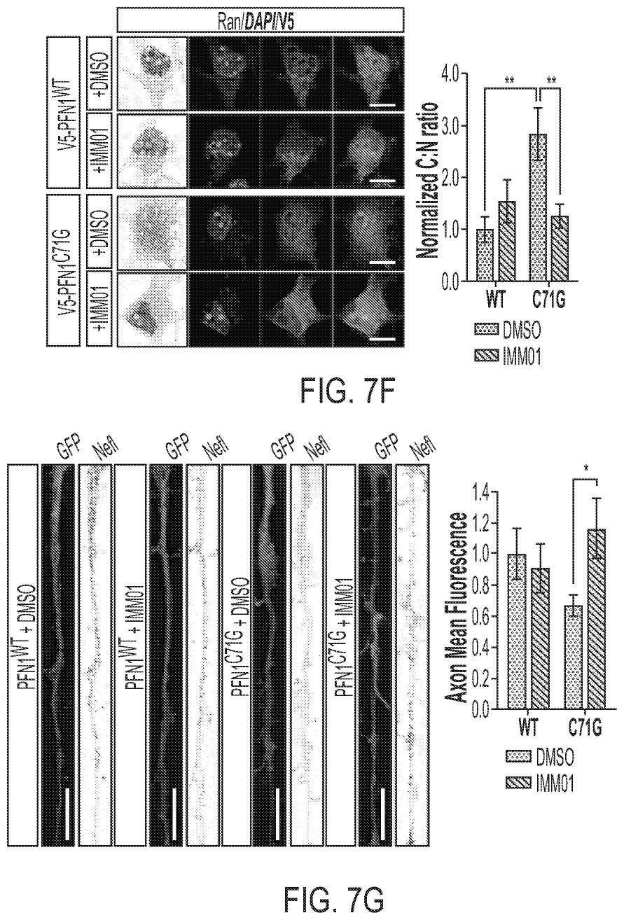

The primary cellular function of PFN1 is to promote actin polymerization by facilitating formin-based actin nucleation and elongation. A severe mislocalization of RanGAP1, FG-Nups, and Ran in MNs treated with the actin depolymerizing drug Latrunculin A (LatA) was observed (FIG. 7A-B), resembling the phenotypes identified in mutant PFN1 MNs. LatA-treated MNs also had smaller nuclei with condensed DNA, demonstrating the disruption of the actin cytoskeleton interfered with normal nuclear morphology. No increased cell death or apoptosis was observed under these treatment conditions (data not shown). To further this observation, a complementary approach was used to rescue NPC defects in C71G PFN1 MNs by positively modulating actin polymerization. Formins promoted actin polymerization, albeit at a slower rate, even in the absence of functional PFN1. Overexpression of a constitutively active form of the formin mDia1 (FH1-FH2 domains), herein referred to as “mDia1”, in mutant PFN1 MNs was able to restore normal actin homeostasis (data not shown), without changing the aggregation propensity of C71G PFN1. While mutant PFN1 MNs expressing GFP alone had significantly higher frequency of disrupted RanGAP1 staining, as previously observed (see FIG. 1), the expression of GFP-mDia1 fully rescued the defect (FIG. 7C). The amino acid sequence of GFP-mDia1 is shown below (SEQ ID NO: 11); the FH1-FH2 domains are underlined. Similarly, GFP-mDia1 expression was able to rescue TDP-43 mislocalization (FIG. 7D) in mutant PFN1 MNs. As a parallel approach, actin polymerization was induced using the small molecule Intramimic-01 (IMM01). Treatment of primary MNs with low concentrations of IMM01 (e.g., 0.1 μM) for 24 hours caused the rescue of FG-Nups defective localization at the nuclear envelope, as well as of the Ran gradient (FIGS. 7E-7F). No induction of apoptosis was detected under these conditions. Importantly, it was found that cytoskeletal remodeling induced by IMM01 treatment was also able to rescue the defects in the axonal localization for the Nelf mRNA (FIG. 7G).

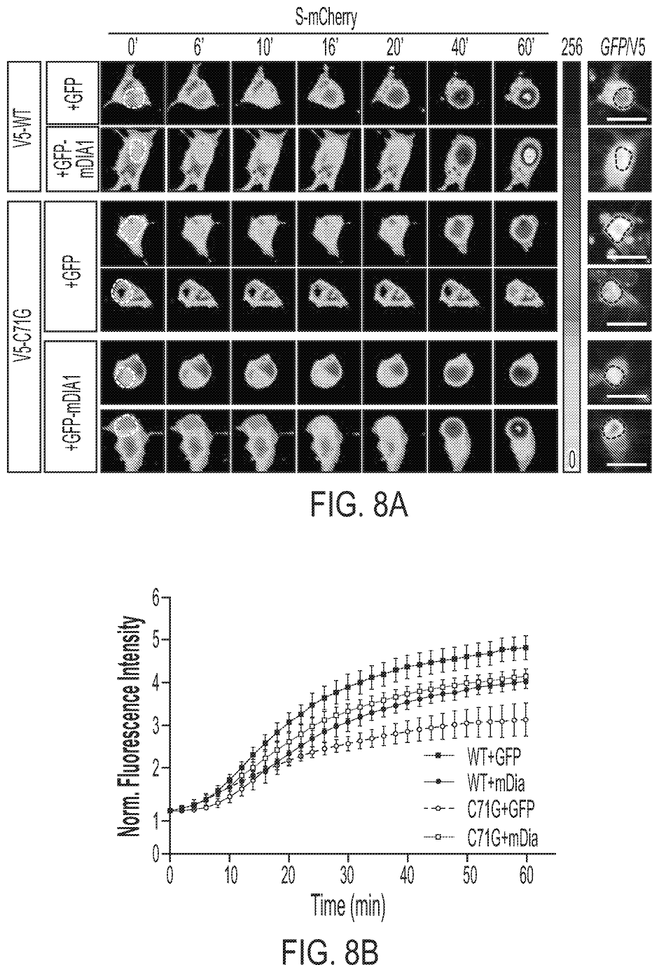

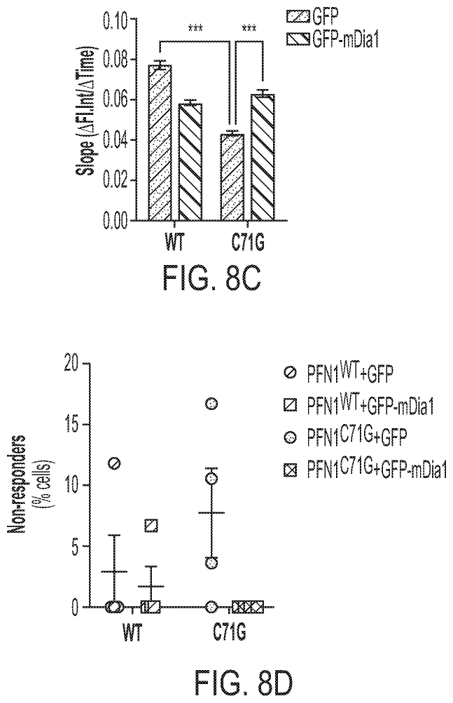

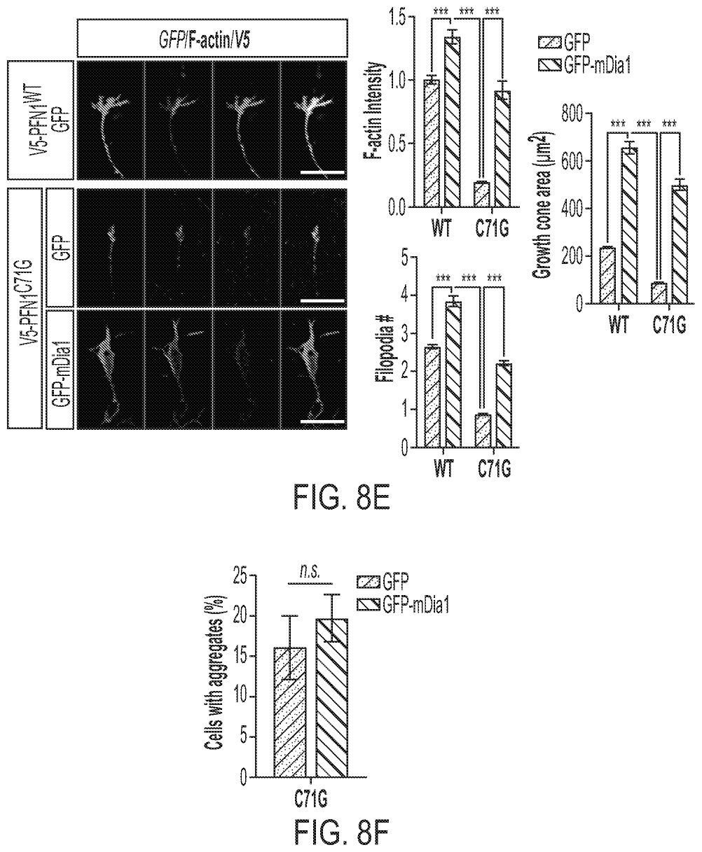

To assess whether actin modulation could also rescue the function of the nuclear pore, import dynamics of S-mCherry were measured in the presence of mDia1 overexpression. Import dynamics were increased in C71G PFN1 MNs following mDia1 expression (FIGS. 8A-8F), showing that changes in actin homeostasis can influence the stability of the NPC and/or NE, affecting protein shuttling. Together, these data show a direct link between actin polymerization, NCT, and mRNA post-transcriptional regulation, and demonstrate that these pathways are central to the onset and progression of the degenerative process in diseases having NCT defects (such as PFN1-linked ALS).

GFP-mDia (Formin FH1-FH2 domains are
underlined)
(SEQ ID NO: 11)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYKSGLRSRMASLS
AVVVAPSVSSSAAVPPAPPLPGDSGTVIPPPPPPPPLPGGVVPPSPPLPP
GTCIPPPPPLPGGACIPPPPQLPGSAAIPPPPPLPGVASIPPPPPLPGAT
AIPPPPPLPGATAIPPPPPLPGGTGIPPPPPPLPGSVGVPPPPPLPGGPG
LPPPPPPFPGAPGIPPPPPGMGVPPPPPFGFGVPAAPVLPFGLTPKKVYK
PEVQLRRPNWSKFVAEDLSQDCFWTKVKEDRFENNELFAKLTLAFSAQTK
TSKAKKDQEGGEEKKSVQKKKVKELKVLDSKTAQNLSIFLGSFRMPYQEI
KNVILEVNEAVLTESMIQNLIKQMPEPEQLKMLSELKEEYDDLAESEQFG
VVMGTVPRLRPRLNAILFKLQFSEQVENIKPEIVSVTAACEELRKSENFS
SLLELTLLVGNYMNAGSRNAGAFGFNISFLCKLRDTKSADQKMTLLHFLA
ELCENDHPEVLKFPDELAHVEKASRVSAENLQKSLDQMKKQIADVERDVQ
NFPAATDEKDKFVEKMTSFVKDAQEQYNKLRMMHSNMETLYKELGDYFVF
DPKKLSVEEFFMDLHNFRNMFLQAVKENQKRRETEEKMRRAKLAKEKAEK
ERLEKQQKREQLIDMNAEGDETGVMD

### Example 8. Modulation of Actin Polymerization Rescues NCT Defects in C9ORF72-ALS

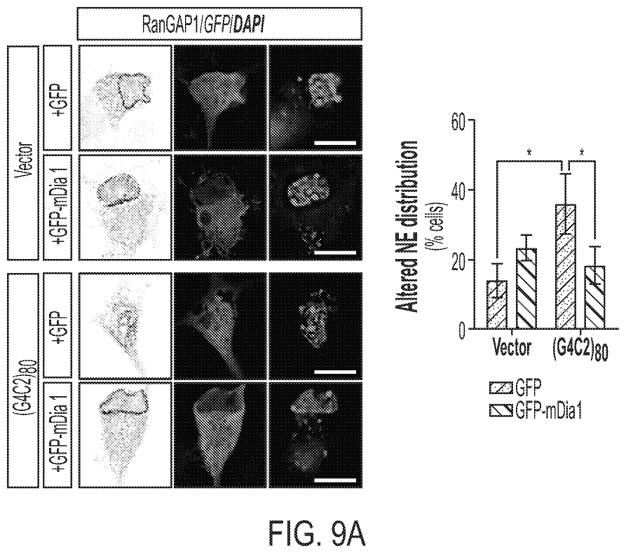

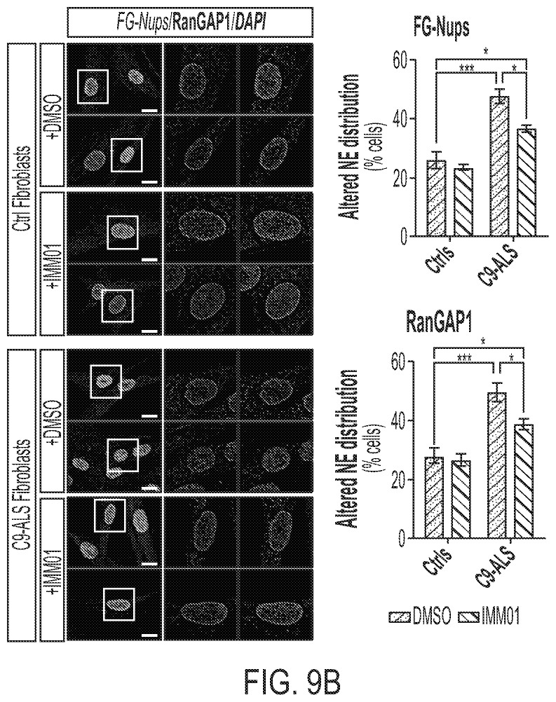

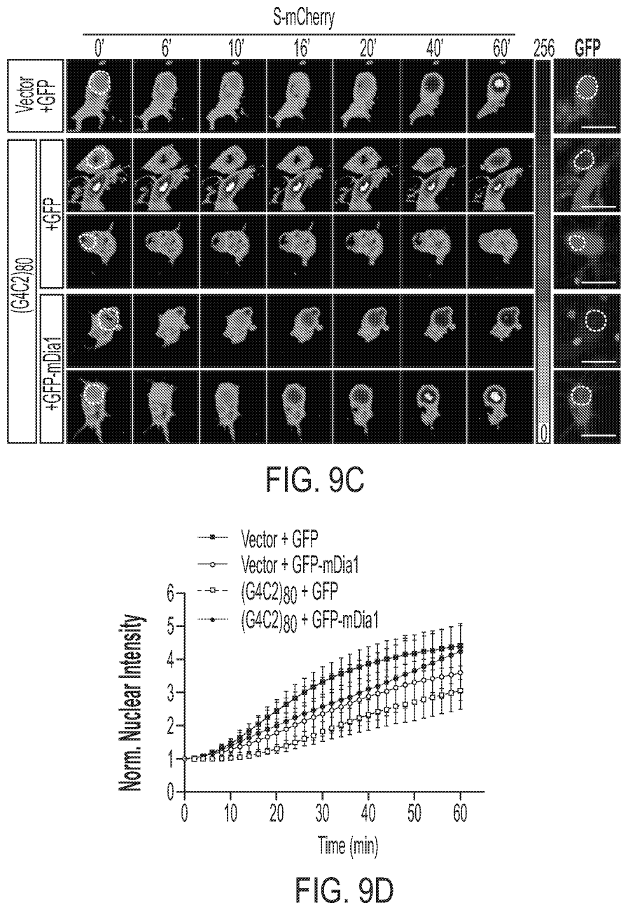

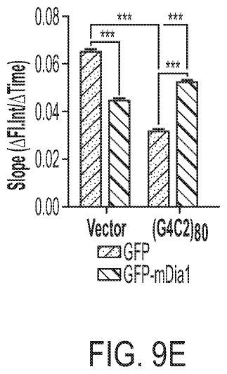

Motor neurons (MNs) were transfected with a synthetic C9ORF72 construct expressing 80 GGGGCC repeats (G₄C₂)₈₀ (SEQ ID NO:12). The expression of this construct did not result in any obvious difference in F-actin levels at the growth cone under experimental conditions. Expression of (G₄C₂)₈₀ (SEQ ID NO:12). repeats in MNs led to the loss of RanGAP1 localization to the NE with no change to its overall levels (FIG. 9A). Overexpression of the constitutively active form of mDia1 fully rescued the phenotype. Fibroblasts obtained from 3 patients carrying the C9ORF72 repeat expansion and 3 healthy controls were treated with the formin activator IMM01. It was found that a higher percentage of the C9ORF72-ALS fibroblasts had disrupted or abnormal staining for both proteins (FIG. 9B). Similarly to what was observed with mDia1 overexpression, IMM01 treatment rescued defects in the localization of RanGAP1 and FG-Nups, without changes to the propensity of the cell to form C9ORF72-positive nuclear foci. mDia1 expression was also able to rescue functional import defects in cortical neurons expressing (G₄C₂)₈₀ (SEQ ID NO:12). repeats, as detected by S-mCherry dynamics (FIG. 9C-9E). Together, these results show that modulation of actin homeostasis directly modifies the stability of the NPC and/or NE, protecting it from disruption caused by ALS-associated gene mutations.

### EQUIVALENTS

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of modulating the function of the nuclear pore in a cell, the method comprising delivering a molecular agent that stabilizes the cytoskeleton to the cell.

2. The method of claim 1, wherein the method comprises modulating nucleocytoplasmic transport (NCT) in the cell.

3. The method of claim 1 or 2, wherein the molecular agent is a molecular agent that promotes actin and/or tubulin polymerization.

4. The method of claim 1 or 2, wherein the molecular agent is a molecular agent that inhibits actin depolymerization.

5. The method of any one of claims 1-4, wherein the cell is a neural cell, optionally wherein the neural cell is a neuroblast, a neural glial cell or a neuron, further optionally wherein the neuron is a motor neuron.

6. The method of any one of claims 1-5, wherein the method rescues actin polymerization, cytoskeletal growth, and/or division in the cell, optionally wherein the method rescues axon growth in a neural cell.

7. The method of any one of claims 1-6, wherein the cell comprises a PFN1 mutation or a repeat expansion in C9ORF72.

8. The method of claim 7, wherein the PFN1 mutation comprises C71G, M114T, G118V, A20T, T109M, Q139L, or E117G.

9. The method of claim 7, wherein the repeat expansion in C9ORF72 comprises at least 80 GGGGCC (G₄C₂) repeats (SEQ ID NO: 12).

10. The method of any one of claims 1-9, wherein the molecular agent is a transgene, protein, or a small molecule.

11. The method of claim 10, wherein the transgene encodes a protein.

12. The method of claim 10 or 11, wherein the protein is an enzyme that polymerizes actin, an actin-severing protein, an actin capping protein, or an actin bundling protein.

13. The method of claim 12, wherein the enzyme that polymerizes actin is a formin, a profilin-1 (PFN1), a profilin-2 (PFN2), an Arp2/3 complex, an Ena/VASP homology protein, or a Wiskott-Aldrich syndrome protein.

14. The method of claim 13, wherein the formin is a constitutively active formin.

15. The method of claim 13 or 14, wherein the formin minimally comprises an FH1 domain and an FH2 domain.

16. The method of claim 12, wherein the actin-severing protein is a cofilin or a variant thereof.

17. The method of claim 12, wherein the actin capping protein is a tropomodulin or a variant thereof.

18. The method of claim 12, wherein the actin bundling protein is a filamin, a fimbrin or a variant thereof.

19. The method of any one of claims 10-18, wherein the transgene is delivered using a viral vector, an antibody-drug conjugate (ADC), closed ended DNA (ceDNA), or messenger RNA (mRNA).

20. The method of any one of claims 10-18, wherein the transgene is delivered using a recombinant adeno-associated virus (rAAV).

21. The method of claim 20, wherein the rAAV comprises:
(a) a capsid protein; and,
(b) a nucleic acid comprising a promoter operably linked to the transgene.

22. The method of claim 21, wherein the capsid protein has an AAV9 serotype.

23. The method of any one of claims 18-22, wherein the delivery results in expression of the transgene in the cell.

24. The method of claim 10, wherein the small molecule is IMM-01, paclitaxel, swinholide, jasplakinolide, or phalloidin.

25. The method of any one of claims 1-24, wherein modulating the function of the nuclear pore comprises modulating the activity, expression, or localization of a nucleoporin of the FG-Nup family, Nup358/RanBP2, POM121, RanGAP1, an importin, an exportin, and/or a RNA-binding protein.

26. The method of claim 25, wherein the nucleoporin of the FG-Nup family is Nup62, Nup153, Nup214, or Nup358.

27. The method of claim 25, wherein the importin is Importin-β.

28. The method of claim 25, wherein the exportin is XPO1.

29. The method of claim 25, wherein the RNA-binding protein is TDP-43, FUS, SMN, or FMRP.

30. The method of any one of claims 1-29, wherein modulating the function of the nuclear pore leads to increased transport of proteins and/or nucleic acids across the nuclear membrane, optionally wherein increased transport is increased nuclear import.

31. The method of any one of claims 1-29, wherein modulating the function of the nuclear pore leads to decreased nuclear export.

32. A method of increasing actin polymerization in a cell, the method comprising delivering a nucleic acid comprising a transgene that encodes a formin, wherein the formin comprises an FH1 domain and FH2 domain.

33. The method of claim 32, wherein the formin is a constitutively active formin.

34. The method of claim 32 or 33, wherein the formin minimally comprises an FH1 domain and an FH2 domain.

35. The method of any one of claims 32-34, wherein the transgene is delivered using a viral vector, an antibody-drug conjugate (ADC), closed ended DNA (ceDNA), or messenger RNA (mRNA).

36. The method of any one of claims 32-35, wherein the transgene is delivered using a recombinant adeno-associated virus (rAAV).

37. The method of claim 36, wherein the rAAV comprises:
(a) a capsid protein; and,
(b) a nucleic acid comprising a promoter operably linked to the transgene.

38. The method of claim 37, wherein the capsid protein has an AAV9 serotype.

39. The method of any one of claims 32-38, wherein the delivery results in expression of the transgene in the cell.

40. A method of treating a subject having a neurodegenerative disease, the method comprising administering a molecular agent that stabilizes the cytoskeleton to the subject.

41. The method of claim 40, wherein the method comprises administering a molecular agent that promotes actin and/or tubulin polymerization.

42. The method of claim 40, wherein the method comprises administering a molecular agent that inhibits actin and/or tubulin depolymerization.

43. The method of claim 40, wherein the neurodegenerative disease is Amyotrophic lateral sclerosis (ALS), Alzheimer's disease, Huntington's disease, or Frontotemporal dementia (FTD), optionally wherein the neurodegenerative disease is associated with a nucleocytoplasmic transport (NCT) defect.

44. The method of claim 43, wherein the subject having ALS has sporadic ALS or familial ALS.

45. The method of claim 43 or 44, wherein the subject has a mutation in at least one gene or protein selected from the group consisting of: C9ORF72, PFN1, TUBA4A, KIF5A TDP 43, SOD1, kinesin, and Tau.

46. The method of claim 45, wherein the mutation in PFN1 protein is C71G, M114T, G118V, A20T, T109M, Q139L, or E117G.

47. The method of claim 45, wherein the mutation in C9ORF72 is a repeat expansion, optionally wherein the repeat expansion comprises 80 GGGGCC repeats ((G₄C₂)₈₀) (SEQ ID NO: 12).

48. The method of any one of claims 40-47, wherein administering the molecular agent that stabilizes the cytoskeleton leads to proper regulation of the nuclear pore.

49. The method of any one of claims 40-48, wherein administering the molecular agent that stabilizes the cytoskeleton leads to increased transport of proteins and/or nucleic acids across the nuclear membranes of cells of the central nervous system, optionally wherein increased transport is increased nuclear import.

50. The method of any one of claims 40-49 wherein the molecular agent is a transgene, protein, or a small molecule.

51. The method of claim 50, wherein the transgene encodes a protein.

52. The method of claim 50 or 51, wherein the protein is an enzyme that polymerizes actin, an actin-severing protein, an actin capping protein, or an actin bundling protein.

53. The method of claim 52, wherein the enzyme that polymerizes actin is a formin, a profilin-1 (PFN1), a profilin-2 (PFN2), an Arp2/3 complex, an Ena/VASP homology protein, or a Wiskott-Aldrich syndrome protein.

54. The method of claim 53, wherein the formin is a constitutively active formin.

55. The method of claim 53 or 54, wherein the formin minimally comprises an FH1 domain and an FH2 domain.

56. The method of claim 52, wherein the actin-severing protein is a cofilin or a variant thereof.

57. The method of claim 52, wherein the actin capping protein is a tropomodulin or a variant thereof.

58. The method of claim 52, wherein the actin bundling protein is a filamin, a fimbrin or a variant thereof.

59. The method of any one of claims 50-58, wherein the transgene is delivered using a viral vector, an antibody-drug conjugate (ADC), closed ended DNA (ceDNA), or messenger RNA (mRNA).

60. The method of any one of claims 50-58, wherein the transgene is delivered using a recombinant adeno-associated virus (rAAV).

61. The method of claim 60, wherein the rAAV comprises:
(a) a capsid protein; and,
(b) a nucleic acid comprising a promoter operably linked to the transgene.

62. The method of claim 61, wherein the capsid protein has an AAV9 serotype.

63. The method of any one of claims 59-62, wherein the transgene is administered via injection, optionally wherein the injection is selected from the group consisting of intravenous injection, intravascular injection and intraventricular injection.

64. The method of any one of claims 59-63, wherein the administration results in expression of the transgene in the central nervous system tissue and/or the peripheral tissue of the subject.

65. The method of claim 50, wherein the small molecule is IMM-01, paclitaxel, swinholide, jasplakinolide, or phalloidin.

66. The method of any one of claims 40-65, wherein the method comprises modulating the activity, expression, or localization of a nucleoporin of the FG-Nup family, Nup358/RanBP2, POM121, RanGAP1, an importin, an exportin, and/or a RNA-binding protein in a cell of the central nervous system of the subject.

67. The method of claim 66 wherein the nucleoporin of the FG-Nup family is Nup62, Nup153, Nup214, or Nup358.

68. The method of claim 66, wherein the importin is Importin-β.

69. The method of claim 66, wherein the exportin is XPO1.

70. The method of claim 66, wherein the RNA-binding protein is TDP-43, FUS, SMN, or FMRP.
